SEQUENCE LISTING (15 sequences)

SEQ ID NO: 1 - DNA, 22 bp, Artificial Sequence
  forward primer
acaccgtttctatagattagct

SEQ ID NO: 2 - DNA, 21 bp, Artificial Sequence
  backward primer
ggcaattttgttaccatcagt

SEQ ID NO: 3 - DNA, 46 bp, Artificial Sequence
  forward inner primer
ggttccacctggtttaacatatagtgtgtgctcaagtattgagtga

SEQ ID NO: 4 - DNA, 46 bp, Artificial Sequence
  backward inner primer
caggagatgccacaactgcttatagataaaagtgcattaacattgg

SEQ ID NO: 5 - DNA, 19 bp, Artificial Sequence
  forward primer
accgaagagctaccagacg

SEQ ID NO: 6 - DNA, 20 bp, Artificial Sequence
  backward primer
tgcagcattgttagcaggat

SEQ ID NO: 7 - DNA, 40 bp, Artificial Sequence
  forward inner primer
tctggcccagttcctaggtagttcgtggtggtgacggtaa

SEQ ID NO: 8 - DNA, 40 bp, Artificial Sequence
  backward inner primer
agacggcatcatatgggttgcacgggtgccaatgtgatct

SEQ ID NO: 9 - DNA, 19 bp, Artificial Sequence
  forward primer
agctgatgagtacgaactt

SEQ ID NO: 10 - DNA, 22 bp, Artificial Sequence
  backward primer
ttcagatttttaacacgagagt

SEQ ID NO: 11 - DNA, 44 bp, Artificial Sequence
  forward inner primer
accacgaaagcaagaaaaagaagtattcgtttcggaagagacag

SEQ ID NO: 12 - DNA, 44 bp, Artificial Sequence
  backward inner primer
ttgctagttacactagccatccttaggttttacaagactcacgt

SEQ ID NO: 13 - DNA, 28 bp, Artificial Sequence
  probe
ttttttttttctacaacacgttgtatgt

SEQ ID NO: 14 - DNA, 28 bp, Artificial Sequence
  probe
ttttttttttaagatcaggaactctaga

SEQ ID NO: 15 - DNA, 28 bp, Artificial Sequence
  probe
ttttttttttccttgaggaagttgtagc

(19) United States
(12) Patent Application Publication
(10) Pub. No.: US 2023/0226543 A1
(43) Pub. Date: Jul. 20, 2023

(54) MICROFLUIDIC DEVICE FOR SARS-COV-2 DETECTION AND METHOD USING THE SAME

(71) Applicant: National Tsing Hua University, Hsinchu City (TW)

(72) Inventors: Gwo-Bin Lee, Hsinchu City (TW); Chih-Hung Wang, Hsinchu City (TW); You-Ru Jhou, Hsinchu City (TW); Yu-Shiuan Tsai, Hsinchu City (TW)

(21) Appl. No.: 17/580,364

(22) Filed: Jan. 20, 2022

(51) Int. Cl. B01L 3/00; C12Q 1/70; C12Q 1/6844; B01L 7/00

(52) U.S. Cl. CPC B01L 3/502715; B01L 3/50273; B01L 3/502738; B01L 3/502753; B01L 7/52; C12Q 1/701; C12Q 1/6844; B01L 2200/16; B01L 2300/18; B01L 2300/0654; B01L 2300/0819; B01L 2300/0867; B01L 2400/06; B01L 2400/043; B01L 2400/0478; G01N 2800/26

(57) ABSTRACT

Provided is an integrated microfluidic device for SARS-CoV-2 detection. Also provided is a method for detecting SARS-CoV-2 by using the same, comprising viral lysis, RNA extraction, and reverse-transcription loop-mediated isothermal amplification (RT-LAMP). The integrated microfluidic device of the present disclosure is small in size, automatically operatable, and easy to use by ordinary people, and the present disclosure can achieve rapid detection with high sensitivity and specificity.

### TECHNICAL FIELD

The present disclosure relates to pathogen detection, and more particularly to detection of severe acute respiratory syndrome coronavirus 2 (SARS-CoV-2).

### DESCRIPTION OF RELATED ART

The coronavirus diseases 2019 (COVID-19), which began in late 2019, was caused by an RNA virus, SARS-CoV-2. The outbreak of SARS-CoV-2 soon became a global pandemic recognized by World Health Organization. Rapid and accurate diagnostic methods can effectively prevent the spread of infections, and the critical point to achieving this goal is the development of SARS-CoV-2 detection devices and methods.

One of the most well-known virus detection methods in the art is reverse-transcription polymerase chain reaction (RT-PCR), which amplifies viral RNA to a large enough amount and is highly sensitive and specific. However, RT-PCR is time-consuming (approximately 2 to 4 hours), labor-intensive, and must be performed by well-trained technicians. As an alternative, other detection methods, such as virus antigen and antibody testing are also used. Such methods are relatively fast and simple, but they lack the specificity and sensitivity of nucleic acid-based approaches and may generate false-positive or false-negative results. Also, antibodies are only observed in blood in the middle and later stages of the infection and thus are not suitable for detection at the early stage.

Several devices for SARS-CoV-2 detection have been reported. However, these devices are not fully automated (i.e., manual intervention is required during the detection process) and may need to be operated by well-trained technicians. Moreover, the sensitivity and specificity of these devices need to be further improved. In addition, not only qualitative detection of the virus is required, but also quantitative detection on the device.

Therefore, there is still an unmet need in the art to develop a device and a method for SARS-CoV-2 detection to solve the above problems.

### SUMMARY

In view of the foregoing, the present disclosure provides an integrated microfluidic device for SARS-CoV-2 detection, comprising a microfluidic chip, a flow control module, and a temperature control module. The microfluidic chip has a plurality of chambers for loading a sample, a reagent, a buffer, or a mixture thereof, wherein the chambers comprise a plurality of first functional chambers containing a loop-mediated isothermal amplification (LAMP) composition, and the LAMP composition in each of the first functional chambers comprises primers of SEQ ID NO. 1 to NO. 4, primers of SEQ ID NO. 5 to NO. 8, or primers of SEQ ID NO. 9 to NO. 12, as listed in Table 1. The flow control module is configured for transporting the sample, the reagent, the buffer, or the mixture thereof between the chambers. The temperature control module is configured for controlling and/or keeping a temperature during a reaction.

In at least one embodiment of the present disclosure, the first functional chambers contain primers of SEQ ID NOs. 1 to 4, primers of SEQ ID NOs. 5 to 8, and primers of SEQ ID NOs. 9 to 12. In some embodiments, at least three of the first functional chambers contain primers of SEQ ID NOs. 1 to 4, primers of SEQ ID NOs. 5 to 8, and primers of SEQ ID NOs. 9 to 12, respectively.

In at least one embodiment of the present disclosure, a temperature control module is configured for controlling and/or keeping the temperature during the reaction of LAMP at a temperature of from 60° C. to 65° C., e.g., 60° C., 61° C., 62° C., 63° C., 64° C., or 65° C. In some embodiments, the temperature during the reaction of LAMP is preferably 60° C.

In at least one embodiment of the present disclosure, the microfluidic chip further comprises one or more second functional chambers for loading the sample and/or conducting viral lysis. In some embodiments, the temperature control module is configured for controlling and/or keeping the temperature during the reaction of viral lysis at room temperature or at a temperature of 95° C. ± 5° C. (i.e., 90° C. to 100° C.).

In at least one embodiment, the microfluidic chip further comprises one or more third functional chambers for RNA extraction. In some embodiments, the third functional chamber contains an RNA capture reagent. In some embodiments, the RNA capture reagent is coated with an RNA probe. In some embodiments, the RNA probe is selected from the group consisting of SEQ ID NO. 13, SEQ ID NO. 14, and SEQ ID NO. 15, as listed in Table 2. In some embodiments, the RNA capture reagent is a magnetic bead coated with an RNA probe. In at least one embodiment, the magnetic beads in each of the third functional chambers are respectively coated with RNA probes of SEQ ID NO. 13, SEQ ID NO. 14, or SEQ ID NO. 15, as listed in Table 2. In some embodiments, the temperature control module is configured for controlling and/or keeping the temperature during the reaction of RNA extraction at a temperature of 45° C. ± 5° C. (i.e., 40° C. to 50° C.).

In at least one embodiment of the present disclosure, the temperature control module comprises a relay, a thermoelectric cooler, and a thermocouple. In some embodiments, the relay is configured to turn on the thermoelectric cooler for heating or to turn off the thermoelectric cooler for cooling.

In at least one embodiment of the present disclosure, the microfluidic chip further comprises one or more fourth functional chambers with a micropump for mixing. In at least one embodiment of the present disclosure, the microfluidic chip further comprises one or more microvalves arranged between any two adjacent ones of the chambers.

In at least one embodiment of the present disclosure, the flow control module is a magnetic control module. In some embodiments, the magnetic control module comprises one or more permanent magnets and electromagnets. In some embodiments, the electromagnet and permanent magnet are respectively set on both sides of the micropump and microvalve.

In at least one embodiment of the present disclosure, the flow control module is a pneumatic combined electromagnetic control module. In some embodiments, the pneumatic combined electromagnetic control module comprises a vacuum pump, a compressor, and an electromagnetic valve. In some embodiments, the microfluidic chip further comprises one or more air holes for air controlled by the pneumatic combined electromagnetic control module.

In at least one embodiment of the present disclosure, the LAMP composition further comprises a fluorescent dye. In some embodiments, the fluorescent dye is selected from the group consisting of calcein, SYBR Green I, PicoGreen, EvaGreen, SYTO-80, SYTO-81, SYTO-82, SYTO-83, SYTO-84, SYTO-85, and SYTOX. In some embodiments, the fluorescent dye is preferably calcein.

In at least one embodiment of the present disclosure, the integrated microfluidic device further comprises an optical detection module for exciting the fluorescent dye to generate a fluorescence signal and detecting the fluorescence signal. In some embodiments, the optical detection module comprises a light source, an objective lens, and a photomultiplier tube.

The present disclosure further provides a method for detecting SARS-CoV-2 by the integrated microfluidic device of the present disclosure as mentioned above, comprising: loading a sample into the chambers of the integrated microfluidic device; and conducting the loop-mediated isothermal amplification (LAMP). In at least one embodiment of the present disclosure, all steps after loading the sample into the chambers are automatically operated by the flow control module and/or the temperature control module.

In at least one embodiment of the present disclosure, the sample is loaded in the second functional chamber. In some embodiments, the method further comprises conducting viral lysis in the second functional chamber to obtain a lysis product containing RNA. In some embodiments, the viral lysis is conducted at room temperature or at a temperature of 95° C. ± 5° C. (i.e., 90° C. to 100° C.).

In at least one embodiment of the present disclosure, the method further comprises dividing the lysis product into multiple parts (e.g., 3 parts) and mixing each of the multiple parts of the lysis product with the LAMP composition comprising primers of SEQ ID NOs. 1 to NOs. 4, primers of SEQ ID NOs. 5 to 8, or primers of SEQ ID NOs. 9 to 12, as listed in Table 1.

In at least one embodiment, the LAMP is conducted at a temperature of from 60° C. to 65° C., e.g., 60° C., 61° C., 62° C., 63° C., 64° C., or 65° C. In some embodiments, the temperature during the LAMP is preferably 60° C.

In at least one embodiment of the present disclosure, the method further comprises transporting each of the multiple parts of the lysis product to the third functional chambers for RNA extraction before the step of mixing the lysis product with the LAMP composition. In some embodiments, the RNA extraction is conducted by mixing the lysis product with the RNA capture reagent as mentioned above. In some embodiments, the RNA extraction is conducted at a temperature of 45° C. ± 5° C. (i.e., 40° C. to 50° C.) in the third functional chamber.

In at least one embodiment of the present disclosure, the LAMP is conducted in the first functional chamber or the third functional chamber.

In at least one embodiment of the present disclosure, the method further comprises washing the RNA capture reagent after the RNA extraction.

In at least one embodiment, the method further comprises transporting the sample, the LAMP composition, a buffer, and/or water by the flow control module.

In at least one embodiment, the flow control module is the magnetic control module, and the steps of transporting and mixing are controlled by turning on electromagnets to create a magnetic attraction to the permanent magnet and/or turning off the electromagnet to cancel the magnetic attraction to the permanent magnet.

In at least one embodiment, the flow control module is the pneumatic combined electromagnetic control module, and the step of transporting is controlled by producing a positive pressure and a negative pressure by the compressor, the vacuum pump, and the electromagnetic valve.

In at least one embodiment, the method further comprises exciting the fluorescent dye of the LAMP composition to generate a fluorescence signal and detecting the fluorescence signal by the optical detection module of the integrated microfluidic device during or after the step of conducting the LAMP. In some embodiments, the method further comprises quantifying the concentration of the SARS-CoV-2 according to an accumulative curve of the fluorescence signal.

In at least one embodiment, the integrated microfluidic device is used for real-time quantitative detection for SARS-CoV-2. The optical detection module continuously excites and records the fluorescence signal throughout the LAMP process and obtain an accumulative curve of the fluorescence signal. The quantitation is calculated and transformed in accordance with the accumulative curve of the fluorescence signal. In some embodiments, the LAMP is conducted in the first functional chamber or the third functional chamber, and the optical detection module is aimed at it.

In the present disclosure, an integrated microfluidic device and a method using the same are provided for SARS-CoV-2 detection. The integrated microfluidic device of the present disclosure is small in size, automatically operatable, and easy to use by ordinary people. Also, the present disclosure can not only achieve rapid detection with high sensitivity and specificity but also arrive at quantitative detection on the device.

### BRIEF DESCRIPTION OF THE DRAWINGS

The present disclosure can be more fully understood by reading the following descriptions of the embodiments, with reference made to the accompanying drawings.

FIG. 1 shows a schematic diagram of the entire detection process in the integrated microfluidic device in accordance with embodiments of the present disclosure.

FIG. 2 shows a schematic diagram of the relationship and components in the integrated microfluidic device in accordance with embodiments of the present disclosure.

FIGS. 3A and 3B show schematic diagrams of the actuation procedures and components in the flow control module in accordance with embodiments of the present disclosure. FIG. 3A illustrates the electromagnetic control module, and FIG. 3B illustrates the pneumatic combined electromagnetic control module.

FIG. 4 shows a schematic diagram of the actuation procedures and components in the temperature control module in accordance with embodiments of the present disclosure.

FIG. 5 shows a schematic diagram of the actuation procedures and components in the optical detection module in accordance with embodiments of the present disclosure.

FIGS. 6A and 6B show a schematic diagram of the integrated microfluidic device in accordance with Example 1 of the present disclosure.

FIGS. 7A and 7B show Test Example 1 of Example 1 to optimize the calcein dosage as a fluorescent dye.

FIGS. 8A to 8C show Test Example 2 of Example 1 to conduct on-chip sensitivity tests using cDNA from RdRp gene, E gene, and N gene, respectively.

FIGS. 9A to 9C show Test Example 3 of Example 1 to conduct on-chip specificity tests by comparing SARS-CoV-2, Streptococcus pneumoniae, Pseudomonas aeruginosa, Mycobacterium bovis, Influenza B, Klebsiella pneumonia. FIGS. 9A to 9C are results of RdRp gene, E gene, and N gene, respectively.

FIGS. 10A to 10D show Test Example 4 of Example 1 to conduct on-chip sensitivity tests of combined RNA extraction, and RT-LAMP using synthesized RNA. FIGS. 10A to 10C are gel electrophoresis results of RdRp gene, E gene, and N gene, respectively, and FIG. 10D shows fluorescence results detected by the optical detection module.

FIGS. 11A to 11H show Test Example 5 of Example 1 to conduct on-chip sensitivity tests of combined viral lysis, RNA extraction, and RT-LAMP using inactive SARS-CoV-2. FIGS. 11A and 11B are gel electrophoresis results of RdRp gene. FIGS. 11C and 11D are gel electrophoresis results of E gene. FIGS. 11E and 11F are gel electrophoresis results of N gene. FIGS. 11G and 11H show fluorescence results of RdRp gene, E gene, and N gene detected by the optical detection module.

FIGS. 12A to 12G show Test Example 6 of Example 1 to conduct on-chip sensitivity tests of RT-LAMP using RNA extracted from clinical samples. FIGS. 12A and 12B are gel electrophoresis results of RdRp gene. FIGS. 12C and 12D are gel electrophoresis results of E gene. FIGS. 12E and 12F are gel electrophoresis results of N gene. FIG. 12G shows fluorescence results of RdRp gene, E gene, and N gene detected by the optical detection module.

FIG. 13 shows a schematic diagram of the integrated microfluidic device in accordance with Example 2 of the present disclosure.

FIGS. 14A to 14C show Test Example 7 of Example 2 to conduct on-chip specificity tests by comparing SARS-CoV-2, Streptococcus pneumoniae, Mycobacterium bovis, Influenza A, Influenza B, and Klebsiella pneumonia. FIGS. 14A to 14C are results of E gene, N gene, and RdRp gene, respectively.

FIGS. 15A to 15D show Test Example 8 of Example 2 to conduct on-chip sensitivity tests of combined RNA extraction and RT-LAMP using synthesized RNA. FIGS. 15A to 15C are gel electrophoresis results of E gene, N gene, and RdRp gene, respectively, and FIG. 15D shows fluorescence results detected by the optical detection module.

FIGS. 16A to 16D show Test Example 9 of Example 2 to conduct on-chip sensitivity tests of combined viral lysis, RNA extraction, and RT-LAMP using inactive SARS-CoV-2. FIGS. 16A to 16C are gel electrophoresis results of E gene, N gene, and RdRp gene, respectively, and FIG. 16D shows fluorescence results of E gene, N gene, and RdRp gene detected by the optical detection module.

FIGS. 17A to 17D show Test Example 10 of Example 2 to conduct on-chip sensitivity tests of RT-LAMP using RNA extracted from clinical samples. FIGS. 17A to 17C are gel electrophoresis results of E gene, N gene, and RdRp gene, respectively, and FIG. 17D shows fluorescence results of E gene, N gene, and RdRp gene detected by the optical detection module.

FIGS. 18A to 18C show Test Example 11 of Example 2 to conduct real-time quantification. FIG. 18A shows a curve between RT-LAMP time and the optical detection signal (response to fluorescence) of RdRp gene from synthesized RNA. FIG. 18B shows standard curves between Log (copy number/time) and the threshold time of E gene, N gene, and RdRp gene from clinical samples. FIG. 18C shows standard curves between Log (copy number/time) and the threshold time of RdRp gene from synthesized RNA, inactive virus, and the clinical samples.

### DETAILED DESCRIPTION

The following embodiments are provided to illustrate the present disclosure in detail. A person having ordinary skill in the art can easily understand the advantages and effects of the present disclosure after reading this disclosure, and also can implement or apply in other different embodiments. Therefore, any element or method within the scope of the present disclosure disclosed herein can combine with any other element or method disclosed in any embodiments of the present disclosure.

The proportional relationships, structures, sizes, and other features shown in accompanying drawings of this disclosure are only used to illustrate embodiments described herein, such that those with ordinary skill in the art can read and understand the present disclosure therefrom, of which are not intended to limit the scope of this disclosure. Any changes, modifications, or adjustments of said features, without affecting the designed purposes and effects of the present disclosure, should all fall within the scope of the technical content of this disclosure.

As used herein, the terms “comprise,” “comprising,” “include,” “including,” “have,” “having,” “contain,” “containing,” and any other variations thereof are intended to cover a non-exclusive inclusion. For example, when describing an object “comprises” a limitation, unless otherwise specified, it may additionally include other elements, components, structures, regions, parts, devices, systems, steps, or connections, etc., and should not exclude other limitations.

As used herein, sequential terms such as “first,” “second,” etc., are only cited in convenience of describing or distinguishing limitations such as elements, components, structures, regions, parts, devices, systems, etc. from one another, which are not intended to limit the scope of this disclosure, nor to limit spatial sequences between such limitations. Further, unless otherwise specified, wordings in singular forms such as “a,” “an” and “the” also pertain to plural forms, and wordings such as “or” and “and/or” may be used interchangeably.

As used herein, the terms “patient” may be interchangeable and refer to an animal, e.g., a mammal including the human species. The term “subject” is intended to refer to both the male and female gender unless one gender is specifically indicated.

As used herein, the phrase “at least one,” in reference to a list of one or more elements, should be understood to mean at least one element selected from any one or more of the elements in the list of elements, but not necessarily including at least one of each element listed within the list of elements and not excluding any combinations of elements in the list of elements. This definition also allows that elements may optionally be present other than the elements identified within the list of elements to which the phrase “at least one” refers, whether related or unrelated to those elements identified. Thus, as a non-limiting example, “at least one of A and B” (or, equivalently, “at least one of A or B,” or, equivalently, “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements).

As used herein, the terms “one or more” and “at least one” may have the same meaning and include one, two, three, or more.

As used herein, the terms “loop-mediated isothermal amplification” and “LAMP” comprise reverse transcripted loop-mediated isothermal amplification (RT-LAMP) and can be used interchangeably. In some embodiments for a DNA sample/reactant, the amplification performed on the detection device of the present disclosure is LAMP. In some embodiments for an RNA sample/reactant, reverse transcription must be performed prior to amplification, so using the term “LAMP” for RNA amplification represents “RT-LAMP.” In contrast with RT-PCR, RT-LAMP can amplify nucleic acids without precisely-controlled thermocycling, thereby reducing detection time compared with RT-PCR. The present disclosure thus integrates LAMP technology with microfluidic technology to achieve rapid detection for SARS-CoV-2 with high sensitivity and specificity in a miniaturized and automated manner.

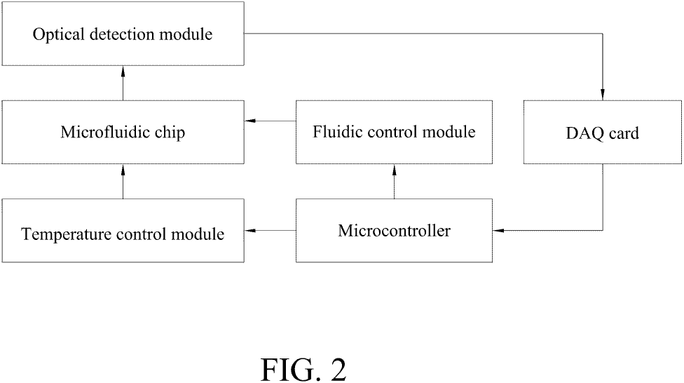

In the present disclosure, an integrated microfluidic device was designed and provided to detect multiple SARS-CoV-2 markers. Coronavirus genomes have 8-10 open reading frames, for instance, ORF1 is translated into 16 non-structural proteins that include the RNA-dependent RNA polymerase enzyme (RdRp). The envelope (E) protein, nucleocapsid (N) protein, spike (S) protein, and membrane proteins are essential for the completion of the viral replication cycle. In at least one embodiment of the present disclosure, the SARS-CoV-2 biomarkers include, but are not limited to, the RNA-dependent RNA polymerase enzyme (RdRp) gene, nucleocapsid (N) gene, and/or envelope (E) gene. As shown in FIG. 2, the integrated microfluidic device comprises a microfluidic chip, a flow control module, and a temperature control module. In some embodiments, the integrated microfluidic device further comprises an optical detection module. The microcontroller may be used to control the flow control module, the temperature module, and/or the optical detection module. The data acquisition (DAQ) card collects signals from measuring units, such as sensors, and sends them to the controller/computer for analysis and processing.

In at least one embodiment of the present disclosure, the microfluidic chip comprises three first functional chambers loading different LAMP compositions for amplifying different genes. Also, the microfluidic chip comprises second functional chambers for loading samples. In some embodiments, the second functional chambers contain viral lysis buffer for lysing SARS-CoV-2 virus samples, so that RNA can be released and used. In some embodiments, for pretreated samples or synthetic samples, such as pre extracted RNA and synthetic RNA, the second functional chambers can be just for loading samples. In at least one embodiment, the microfluidic chip comprises a single second chamber, and samples are divided into multiple parts (e.g., three parts) through channels and microvalves, or the microfluidic chip comprises a plurality of the second chambers. In at least one embodiment, the microfluidic chip further comprises third chambers containing an RNA capture reagent for RNA extraction. In some embodiments, the RNA capture reagent is a magnetic bead coated with specific RNA probes. In the present disclosure, the viral lysis, RNA extraction, and LAMP are carried out at a suitable temperature that is well-controlled by the temperature control module. Samples, reagents, mixtures, and products are transported by the flow control module.

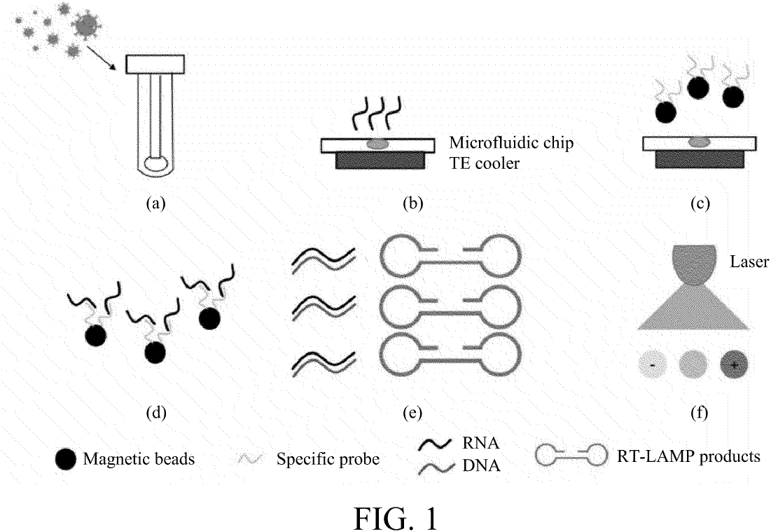

One of the detection protocol using the integrated microfluidic device is shown in FIG. 1. (a) Virus samples are collected from the throat swab and then loaded on the microfluidic chip for (b) viral lysis. (c) Magnetic beads coated with specific probes preloading into the microfluidic chip are used for RNA extraction. After (d) collecting the magnetic beads, RT-LAMP composition is mixed with them, and (e) RT-LAMP is carried out. RT-LAMP product can be used for gel electrophoresis, or optionally, (f) fluorescence detection for the RT-LAMP product could be used for data analysis.

In some embodiments, steps (a) and (b) may be omitted for synthetic or pretreated RNA samples, or steps (a) to (d) may be omitted for synthetic or pretreated RNA samples. In another embodiment, steps (a) to (d) may be omitted, and step (e) may be replaced with a step of carrying out LAMP for cDNA samples.

In the present disclosure, different LAMP compositions refer to LAMP compositions containing different LAMP primer sets (including F3, B3, FIP, and BIP) for the RdRp gene, E gene, and N gene. The design of LAMP primer sets for the RdRp gene, E gene, and N gene is based on the multiple alignments among the nucleotide sequences of SARS-Co-2 (Genebank Accession No. NC_004718), MERS (Middle East Respiratory Syndrome Coronavirus), and SARS-CoV by using the Clustal Omega. The sequences of designed LAMP primers for the target gene of SARS-CoV-2 are listed in Table 1 below.

TABLE 1

Sequences of designed LAMP primers for the target gene of SARS-CoV-2

| SEQ ID NO. | Primer name | Target gene | Nucleotide sequence (5′ ➔3′) |
|---|---|---|---|
| 1 | COVR F3 | RdRp^{a} | ACACCGTTTCTATAGATTAGCT |
| 2 | COVR B3 | | GGCAATTTTGTTACCATCAGT |
| 3 | COVR FIP | | GGTTCCACCTGGTTTAACATATAGTGTGTGCTCAAGTATTGAGTGA |
| 4 | COVR BIP | | CAGGAGATGCCACAACTGCTTATAGATAAAAGTGCATTAACATTGG |
| 5 | COVN F3 | N^{b} | ACCGAAGAGCTACCAGACG |
| 6 | COVN B3 | | TGCAGCATTGTTAGCAGGAT |
| 7 | COVN FIP | | TCTGGCCCAGTTCCTAGGTAGTTCGTGGTGGTGACGGTAA |
| 8 | COVN BIP | | AGACGGCATCATATGGGTTGCACGGGTGCCAATGTGATCT |
| 9 | COVE F3 | E^{c} | AGCTGATGAGTACGAACTT |
| 10 | COVE B3 | | TTCAGATTTTTAACACGAGAGT |
| 11 | COVE FIP | | ACCACGAAAGCAAGAAAAAGAAGTATTCGTTTCGGAAGAGACAG |
| 12 | COVE BIP | | TTGCTAGTTACACTAGCCATCCTTAGGTTTTACAAGACTCACGT |

^{a}RdRp: RNA dependent RNA polymerase;
^{b}N: nucleocapsid;
^{c}E: envelope

In the present disclosure, RNA extraction is based on the specific probes conjugating to the surface of magnetic beads. The sequences of RNA probes for the target gene of SARS-CoV-2 are listed in Table 2 below. Specifically, the end of the RNA probe is modified by amine and conjugated to the surface of magnetic beads via carboxylation with 1-ethyl-3-(3-dimethylaminopropyl) carbodiimide.

TABLE 2

Sequences of RNA probes for the target gene of SARS-CoV-2

| SEQ ID NO | Probe name | Target gene | Nucleotide sequence (5′ ➔3′) |
|---|---|---|---|
| 13 | COVR R3 | RdRp^{a} | TTT TTT TTT TCT ACA ACA CGT TGT ATG T |
| 14 | COVR E3 | E^{b} | TTT TTT TTT TAA GAT CAG GAA CTC TAG A |
| 15 | COVR N3 | N^{c} | TTT TTT TTT TCC TTG AGG AAG TTG TAG C |

^{a}RdRp: RNA dependent RNA polymerase;
^{b}N: nucleocapsid;
^{c}E: envelope

Without intent to limit the scope of the disclosure, exemplary instruments, methods, and their related results according to the embodiments of the present disclosure are given below. It is noted that titles or subtitles may be used in the examples for the convenience of a reader, which in no way should limit the scope of the disclosure. Moreover, certain theories are proposed and disclosed herein; however, in no way they, whether they are right or wrong, should limit the scope of the disclosure so long as the disclosure is practiced according to the disclosure without regard for any particular theory or scheme of action.

### Example 1: An Integrated Microfluidic Device with a Magnetic Control Module

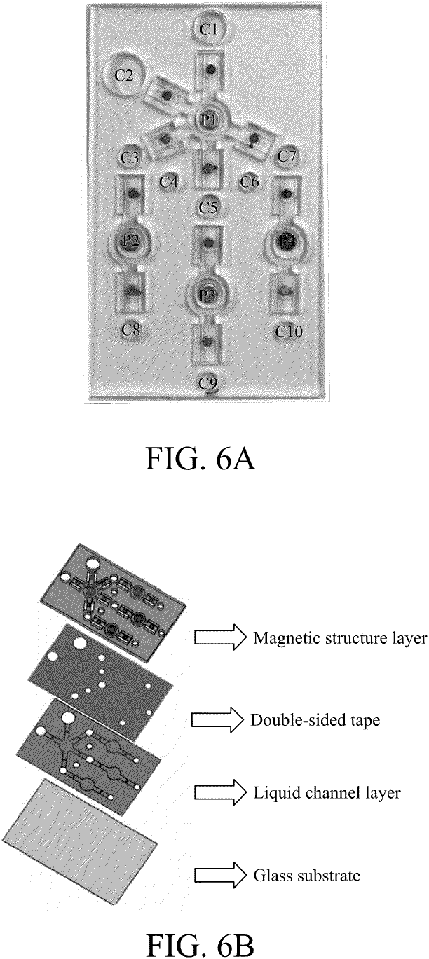

As shown in FIGS. 6A and 6B, a microfluidic chip was composed of 4 layers: a magnetic structure layer, a double-side-tape layer, a liquid channel layer, and a glass substrate (0.4 mm, Ruilong Optoelectronics, Taiwan). The size of the integrated microfluidic device was designed to be 48 × 80 × 3 mm. The magnetic structure layer was used to align detachable magnets with the corresponding electromagnets placed below the microfluidic chip. The liquid channel layer was used for the accommodation and transport of materials, such as samples, reagents, buffer, water, and the mixture thereof.

The magnetic structure layer and the liquid channel layer were microfabricated by casting of polydimethylsiloxane (PDMS; 12:1 A:B ratio) onto polymethyl methacrylate (PMMA) mastermolds. The PDMS was mechanically demolded from PMMA, cured at 80° C. for 5 hours, treated with oxygen plasma (Cute MP/R, Atlas Technology, Taiwan) for 3 minutes, to a glass substrate.

For producing the magnetic structure layer, the permanent magnets were first positioned on a PMMA mold, and PDMS was then poured over the mold. After curing, they were fixed and aligned on the top of PDMS, and the magnetic structure layer was obtained. The magnetic structure layer was then bonded to the liquid channel layer by a double-sided tape. In this step, the liquid channel layer originally had open channels and chambers, and all channels and a part of chambers were closed. The remaining openings were used for loading materials and these materials were accommodated in chambers and could be transported through channels during detection. Finally, the multilayer structure was bonded on a glass substrate to complete the microfluidic chip. Due to the use of the double-sided tape, the permanent magnets could be easily removed from the microfluidic chip and allowed to be reusable.

Specifically, the microfluidic chip in Example 1 comprises a plurality of permanent magnets, microvalves (2.6 × 9 mm), micropumps (6 × 7 mm), chambers, and branched channels. In FIG. 6A, chamber C1 was a second functional chamber for loading sample and carrying out viral lysis; chamber C2 was for loading buffer; C8-C10 were first functional chambers for loading RT-LAMP compositions; C3, C5, and C7 were third functional chambers for loading RNA capture reagent and conducting RNA extraction and RT-LAMP; and C4 and C6 were for negative control and positive control, respectively. Micropumps P1-P4 were arranged in the closed chambers, and microvalves were arranged in the channels between each of the chambers. Permanent magnets were positioned above and aligned with micropumps and microvalves.

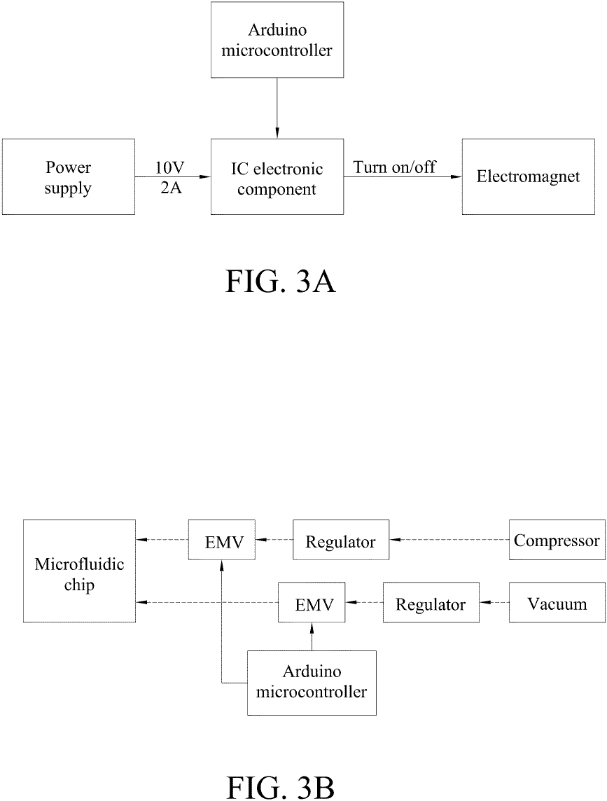

The microfluidic chip was placed on the platform comprising a flow control module, a temperature control module, and optionally further comprising optical detection module. To operate the chip electromagnetically (see FIG. 3A), an Arduino microcontroller (Italy) compiled the codes for controlling the electronic components, and a power supply (CPS-3205 II, Hundred Years Electronic, Taiwan) supplied electric power 8V-10V/2A to the flow control module. The electronic components comprised 9 DC motor bridges (L298n, STMicroelectronics, China) controlling electromagnets made of an iron core (500 coil turns). The electromagnets were positioned below the micropumps and microvalves, corresponding to the permanent magnets. When L298n turned on the electromagnet, the corresponding permanent magnet was magnetically attracted by the electromagnet; and when L298n turned off the electromagnet, the corresponding permanent magnet was not attracted and moved back to the original position. This actuation drove the microvalve between them to close/open the channel and drove the micropumps to move up and down to mix materials. Also, the electromagnets were positioned below the third functional chambers C3, C5, and C7 and drove magnetic beads that served as an RNA capture reagent to move.

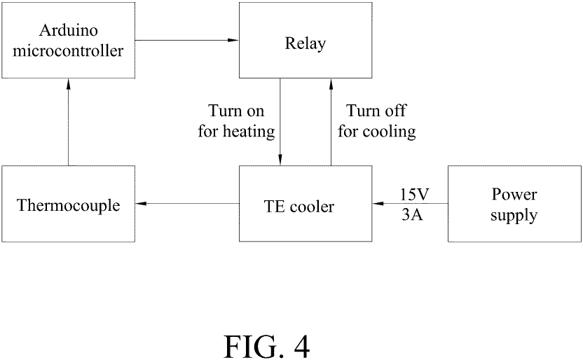

As shown in FIG. 4, the temperature control module was also placed underneath the microfluidic chip, such that the required temperature control could be precisely regulated. The temperature control module comprises a power supply (15V/3A), thermoelectric (TE) cooler (TEC 1-241.10, Tande, Taiwan), a copper plate (Scientific Instrument Center, National Tsing Hua University, Taiwan), two relays (JQC-3FF-S-Z, Hundred Years Electronic) controlled by the Arduino microcontroller, and two thermocouples (Max6675, Hundred Years Electronic). Once the temperature reached the target value, the relays cut off the power to stop heating; on the contrary, when the temperature is too low, the relays turned on the power for heating.

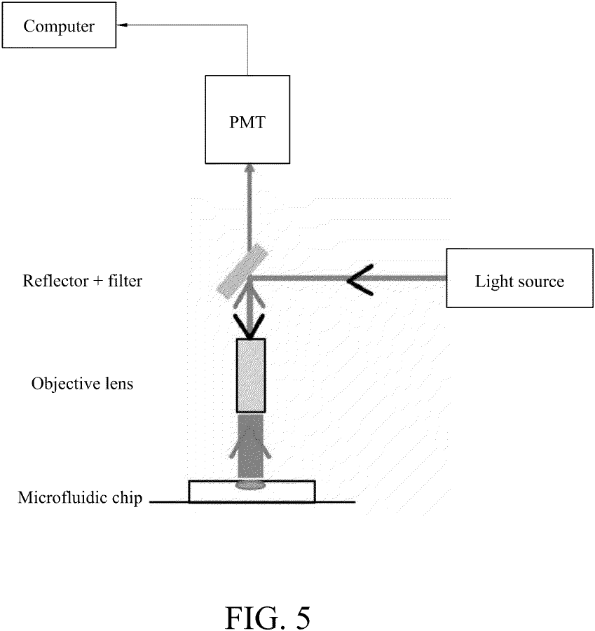

The integrated microfluidic device optionally comprises the optical detection module (see FIG. 5), which comprises a photomultiplier tube (PMT, C6271, Hamamatsu Photonics, Japan) for collecting fluorescence signals, a laser (495 nm, MBL-III-473, Changchun New Industries Optoelectronics, China) for exciting the fluorescent dye (e.g. calcein), a reflector (T495LP, Mirle, China) for reflecting the excited light from the light source to the sample, an optical filter (525 nm, ET525, Chroma ATE, Taiwan), an objective lens (U47365, Onset Electro-optics, Taiwan), and a computer for data analysis.

Before detection, all materials, such as samples, reagents, and buffer, were first prepared and loaded on the microfluidic chip. Table 3 below lists materials pre-loaded on the microfluidic chip when using virus samples.

TABLE 3

Materials loaded on the microfluidic chip

| | | |
|---|---|---|
| Chamber | Materials and volume | Source |
| C1 | 90 µL Lysis buffer | R145, ABP Biosciences, USA |
| | 15µL Sample | |
| C2 | 300 µL Washing buffer | |
| C3 | 5 µL Magnetic beads coated with RNA probes of SEQ ID NO. 15 | 10⁵ beads/µL, 1.05 µm, Invitrogen, USA |
| C4 | 25 µL Negative control | |
| C5 | 5 µL Magnetic beads coated with RNA probes of SEQ ID NO. 14 | 10⁵ beads/µL, 1.05 µm, Invitrogen, USA |
| C6 | 25 µL Positive control | Antech Diagnostics, USA |
| C7 | 5 µL Magnetic beads coated with RNA probes of SEQ ID NO. 13 | 10⁵ beads/µL, 1.05 µm, Invitrogen, USA |
| C8 | 24 µL RT-LAMP composition for N gene | |
| C9 | 24 µL RT-LAMP composition for E gene | |
| C10 | 24 µL RT-LAMP composition for RdRp gene | |

As shown in Table 3, in at least one embodiment of the present disclosure, the process is as follows:
1. The viral RNA lysis buffer (R145, ABP Biosciences, USA, 90 µL) was loaded into chamber C1;
2. The washing buffer (nuclease-free water, 300 µL) was loaded into chamber C2;
3. MyOne™ carboxylic acid magnetic Dynabeads (1.05 µm, Invitrogen, USA) were coated with the RNA probes of SEQ ID NO. 13-15 (modified by amine group) respectively, wherein the coating was based on carboxylation with 1-ethyl-3-(3-dimethylaminopropyl) carbodiimide catalytic action, and these magnetic beads were then loaded into chambers C3, C5 and C7 (10⁵ beads/µL, 5 µL/chamber);
4. The nuclease free water (1 µL) and the RT-LAMP composition (24 µL/chamber) were loaded into chamber C4 as negative control;
5. Synthesized RNAs for RdRp gene, E gene, and N gene (Antech Diagnostics, USA, 10¹³ copies/µL, 1 µL) and the RT-LAMP composition (24 µL/chamber) were loaded into chamber C6 as positive control;
6. The RT-LAMP composition (24 µL/chamber) as shown in Table 4 below was loaded into chamber C8-C10; and
7. After the above loading, the sample (15 µL), selected from synthesized RNAs (for the RdRp gene, E gene and N gene, Antech Diagnostics, USA), synthesized cDNA, inactive viruses (Qnostics, UK) or RNAs extract from clinical samples (National Cheng Kung University Hospital (NCKUH), Taiwan) was loaded into chamber C1.

TABLE 4

RT-LAMP composition

| | | |
|---|---|---|
| Materials | Volume | Source |
| 10 × Isothermal Amplification Buffer II | 2.5 µL | BioLabs Inc. (New England) |
| 10 mM dNTP Mix | 3.0 µL | Promega corp. (USA) |
| 100 mM MgSO₄ | 2.0 µL | BioLabs Inc. (New England) |
| 5 M Betaine | 0.8 µL | BioLabs Inc. (New England) |
| 10 µM primer F3 (SEQ ID NO. 1, 5 or 9) | 0.5 µL | Protech co., Ltd (Taiwan) |
| 10 µM primer B3 (SEQ ID NO. 2, 6, or 10) | 0.5 µL | Protech co., Ltd (Taiwan) |
| 20 µM primer FIP (SEQ ID NO. 3, 7 or 11) | 1.5 µL | Protech co., Ltd (Taiwan) |
| 20 µM primer BIP (SEQ ID NO. 4, 8 or 12) | 1.5 µL | Protech co., Ltd (Taiwan) |
| Bst 3.0 DNA Polymerase (8000 U/ ml) | 1.0 µL | BioLabs Inc. (New England) |
| Reverse transcriptase | 0.5 µL | BioLabs Inc. (New England) |
| Nuclease free water (double distilled water) | 10.2 µL | |
| RNA | 1.0 µL | Antech Diagnostics (USA) |
| TOTAL VOLUME | 25.0 µL | |

For fluorescence detection, 1 µL of 0.5 mM calcein (C0875, Sigma-Aldrich) was added to the RT-LAMP composition in Table 4 and the volume of nuclease free water was adjusted to 9.2 µL.

During the detection, virus samples (e.g. inactive viruses (concentration=10⁴ copies/µL, Qnostics, UK) were firstly lysed in chamber C1 for 20 minutes at room temperature or for 5 minutes at 95° C. through the temperature control module. The lysis product was divided into three equal parts and transported to chambers C3, C5, and C7. RNA extraction was carried out in chamber C3, C5, and C7 via magnetic beads at the same time for 10 minutes at 45° C. Electromagnets were turned on and off alternately to drive magnetic beads to move and mix the product, and the waste liquid part was discarded. The washing buffer was then transported from chamber C2 to chambers C3, C5, and C7 to wash the magnetic beads, followed by discarding the washing buffer. After that, RT-LAMP compositions were transported from chambers C8, C9, and C10 to chambers C3, C5, and C7 respectively, to perform RT-LAMP for 60 minutes at 60° C. The RT-LAMP composition comprises a combination of the forward outer primer (F3), the backward outer primer (B3), the forward inner primer (FIP), and the backward inner primer (BIP) for the RdRp gene, E gene, and N gene. Dumbbell-shaped RT-LAMP products were produced.

The protocol of the detection for virus samples on the microfluidic chip were shown in Table 5 below.

TABLE 5

Protocol of the detection on Example 1

| | | | | |
|---|---|---|---|---|
| Stages | Steps and duration | Reagents and volume | Position | Operating conditions |
| Viral lysis | Thermal lysis for 5 min or chemical lysis for20 min | 105 µL Sample + Lysis buffer | C1 | Voltage for TE cooler: 15 volts 95° C. (thermal lysis) / 0 volt 25° C.(chemical lysis) |
| RNA extraction | a. Division and transport of the lysis product | 105 µL Lysis product ➔ 35 µL Lysis product ➔ 35 µL Lysis product ➔ 35 µL Lysis product | C1 ➔ P1 ➔ C3 C1 ➔ P1 ➔ C5 C1 ➔ P1 ➔ C7 | Voltage for electromagnets, TE cooler: 10 volts/15 volts RNA extraction: 45° C. |
| | b. RNA extraction for 10 min | 35 µL Lysis product + 5 µL Magnetic beads for each gene | C3, C5, and C7 | |
| | c. Liquid waste removal | | C3 ➔ P1 ➔ C1 C5 ➔ P1 ➔ C1 C7 ➔ P1 ➔ C1 | |
| Washing | a. Division and transportation of buffer | 300 µL Nuclease free water ➔ 100 µL Nuclease free water ➔ 100 µL Nuclease free water ➔ 100 µL Nuclease free water | C2 ➔ P1 ➔ C3 C2 ➔ P1 ➔ C5 C2 ➔ P1 ➔ C7 | Voltage for electromagnets:10 volts |
| | b. Liquid waste removal | | C3 ➔ P1 ➔ C1 C5 ➔ P1 ➔ C1 C7 ➔ P1 ➔ C1 | |
| RT-LAMP | a. Transportof RT-LAMP composition | | C8 ➔ P2 ➔ C3 C9 ➔ P3 ➔ C5 C10 ➔ P4 ➔ C7 | Voltage for electromagnets: 10 volts RT-LAMP: 60° C. |
| | b. RT-LAMP for 60 min | 24 µL RT-LAMP composition + Magnetic beads | C3, C4, C5, C6, and C7 | |

On the other hand, for RNA samples (e.g., synthesized RNAs and RNAs extracted from clinical samples), the protocol was to directly perform RT-LAMP; for DNA samples (e.g. synthesized cDNA), the protocol was to omit the stages of viral lysis, RNA extraction, and washing, and perform a stage of LAMP instead of RT-LAMP.

The following Test Example 1 to 6 were used Example 1.

### Test Example 1: Optimizing the Calcein Dosage

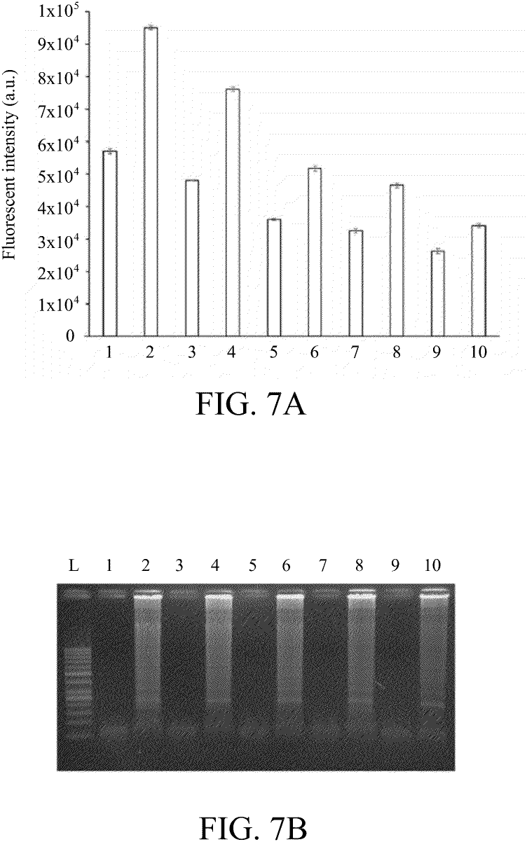

To titrate the optimal ratio of calcein/MnCl₂ to quantify the RT-LAMP products, various concentrations of the MnCl₂ were mixed with 0.5 mM calcein to test and compare the fluorescence intensity between positive control (synthesized RNAs for RdRp gene, 10⁵ copies/reaction) and negative control (nuclease free water). The fluorescence intensity was detected by Enzyme-linked immunosorbent assay (ELISA) reader and the results were shown in FIG. 7A (error bars=standard error of the mean [n=3]), wherein columns 1 and 2 were the group of calcein (0.5 mM) mixed with 5 mM MnCl₂, columns 3 and 4 were the group of calcein mixed with 7.5 mM MnCl₂, columns 5 and 6 were the group of calcein mixed with 10 mM MnCl₂, columns 7 and 8 were the group of calcein mixed with 12.5 mM MnCl₂, and columns 9 and 10 were the group of calcein mixed with 15 mM MnCl₂. Columns 1, 3, 5, 7, and 9 were negative control and columns 2, 4, 6, 8, and 10 were positive control.

A larger difference of fluorescence intensity between positive control and negative control is present on a group consisting of columns 1 and 2 and this result was consistent with gel electrophoresis as shown in FIG. 7B, wherein the sample of each lane in gel electrophoresis corresponded to that of the column with the same number in FIG. 7A. L represented 50-bp DNA ladder.

### Test Example 2: On-Chip LAMP Sensitivity Tests Using Synthesized cDNAs

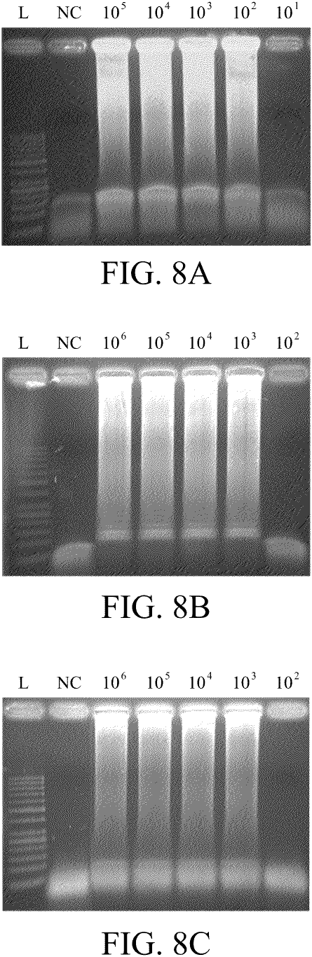

cDNAs reverse-transcribed from RdRp gene, E gene, and N gene were tested at concentrations ranging from 10 to 10⁶ copies/reaction for 60-min LAMP (FIGS. 8A-8C). The LODs for E gene and N gene were found to be 10³ copies/reaction (FIGS. 8B-8C); and the LOD for RdRp gene (FIG. 8A) was only 10² copies/reaction. The polymerase may have lost activity on the other genes, though 10³ copies/reaction is an acceptable LOD for LAMP, and values are often higher in previous works (e.g., 10⁴ copies/reaction). NC represented negative control (nuclease-free water) and L represented 50-bp DNA ladder.

### Test Example 3: On-Chip LAMP Specificity Tests Using Synthesized cDNAs

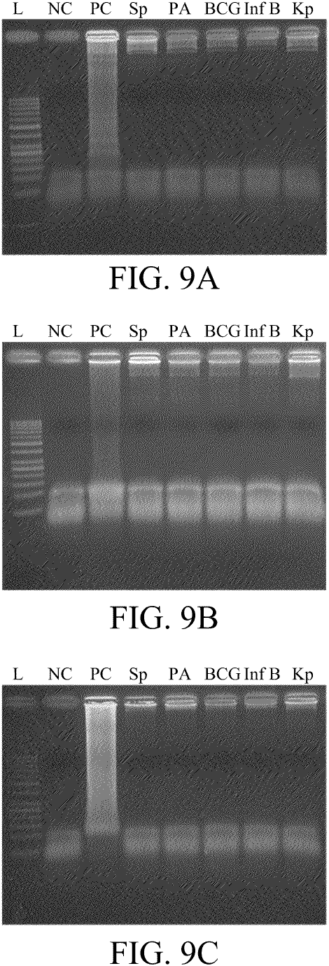

The designed four primers of the present disclosure were also tested for cDNA from the genes of other common acute upper respiratory viruses and bacteria. 7 groups were tested in 60 min LAMP. PC represented synthesized cDNAs from RdRp gene, E gene, and N gene of SARS-CoV-2 respectively in FIGS. 9A-9C at 10³ copies/reaction. Sp represented sample of Streptococcus pneumoniae, PA represented sample from the gene of Pseudomonas aeruginosa, BCG represented sample from the gene of Mycobacterium bovis, Inf B represented sample from the gene of Influenza B, and Kp represented sample from the gene of Klebsiella pneumonia, and the cDNA concentrations of these 5 groups were 10¹¹ copies/reaction. NC represented negative control (nuclease-free water) and L represented 50-bp DNA ladder.

The results showed that the designed primers of the present disclosure are specific for the target SARS-CoV-2.

### Test Example 4: On-Chip RT-LAMP Sensitivity Tests Using Synthesized RNAs

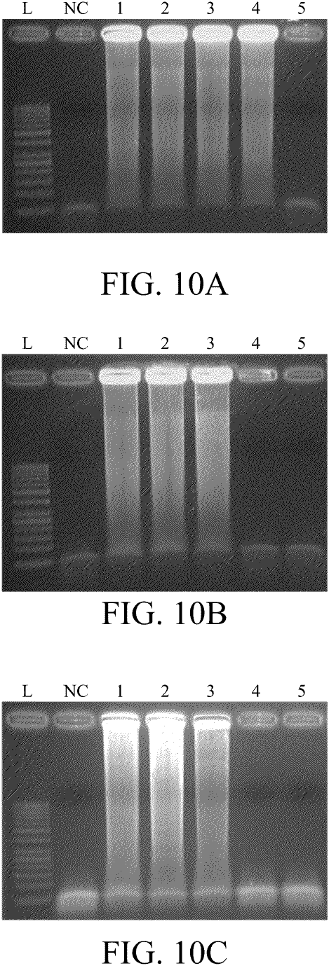

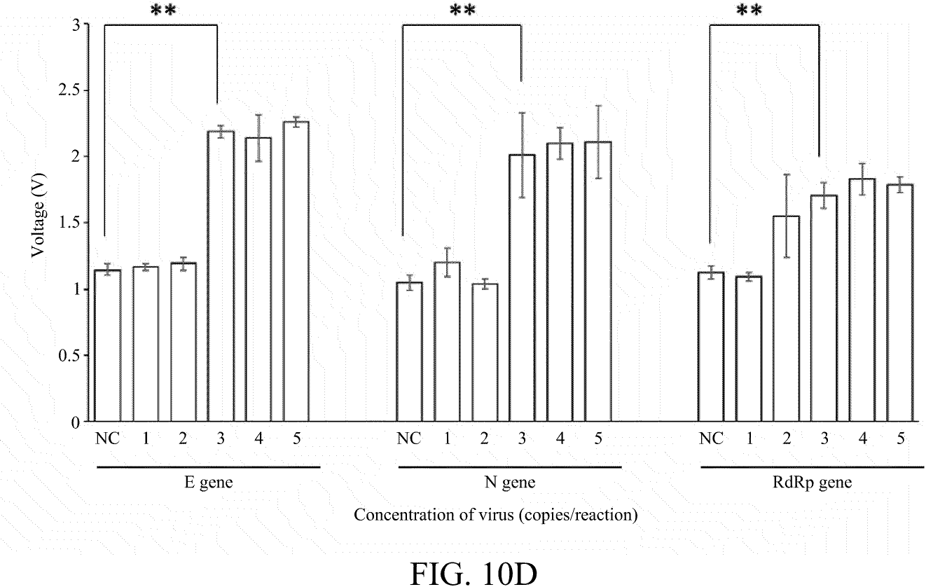

To simulate that viral RNAs were released into solution, 5 µL of synthesized RNAs (ranging from 5 × 10⁵ copies/µL to 5 × 10¹ copies/µL) for the RdRp gene, E gene, and N gene (as shown in FIGS. 10A-10C respectively) were spiked into 30 µL of water and then captured by 5 µL of magnetic beads (10⁶ beads/µL) surface-coated with specific RNA probes (SEQ ID NO. 13-15). Then, RT-LAMP was carried out to explore the sensitivity. The LODs for the E gene and N gene were found to be 5 × 10³ copies/reaction in the combined RNA extraction and RT-LAMP experiment and the LOD for the RdRp gene was only 5 × 10² copies/reaction.

When using the optical detection module (FIG. 10D), the LODs were similar to those of gel electrophoresis, though the LOD for the RdRp gene was slightly increased to the same level as those for E gene, and N gene (5 × 10³ copies/reaction).

These results suggest that the integrated microfluidic device of the present disclosure can successfully detect RNA viruses via RT-LAMP and subsequent fluorescence detection by using the optical detection module.

In FIGS. 10A-10D, NC represented negative control (nuclease-free water) and L represented 50-bp DNA ladder. Lanes/columns 1 to 5 represent 5 × 10⁵ to 5 × 10¹ copies/reaction, respectively. Error bars represented the standard error of the mean (n=3). ** was indicated as p<0.05 by two-tailed student t test analysis.

### Test Example 5: On-Chip Sensitivity Tests Using Inactive Viruses

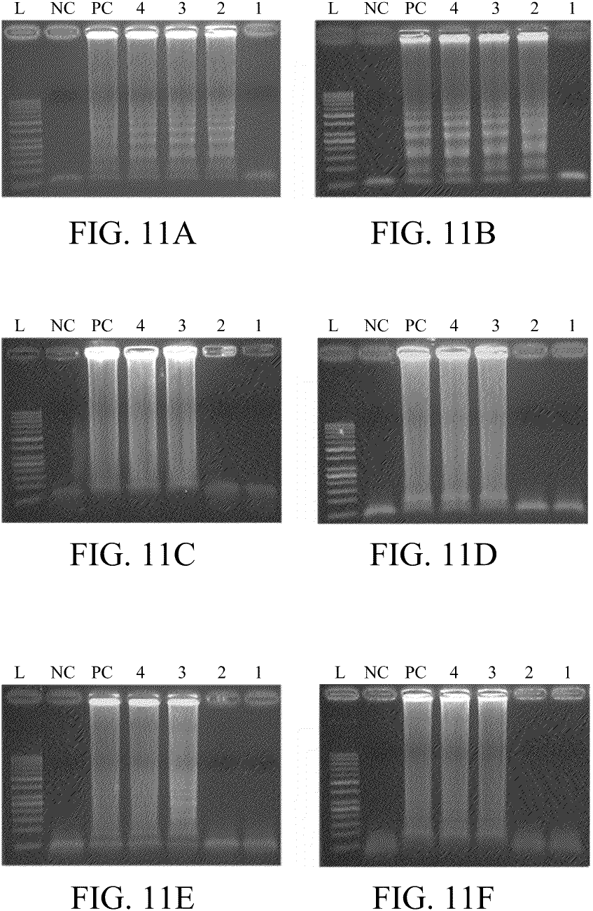

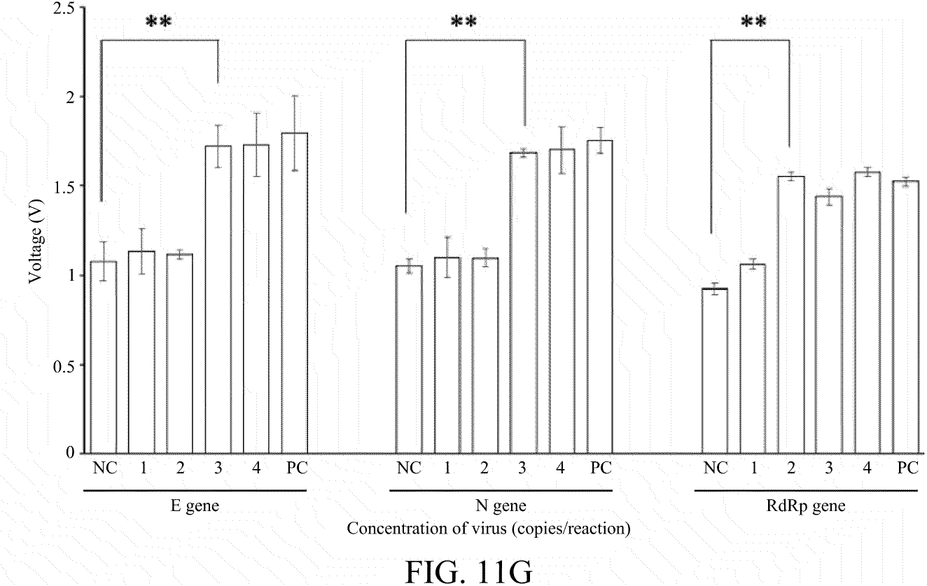

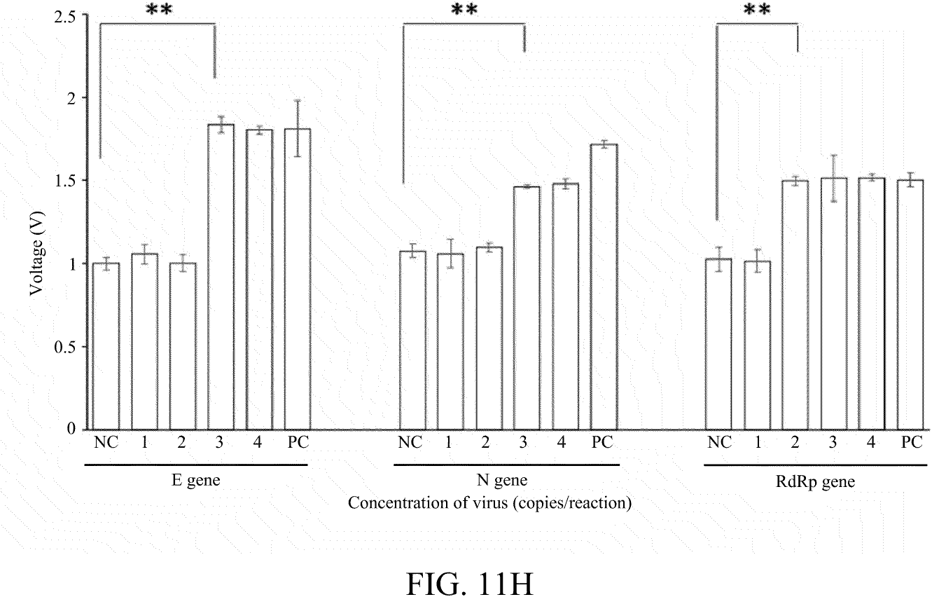

Samples of inactive SARS-CoV-2 viruses were loaded on the microfluidic chip and viral lysis (thermal lysis or chemical lysis), RNA extraction, and RT-LAMP were performed in sequence. The results of sensitivity tests for the RdRp gene, E gene, and N gene were shown by gel electrophoresis (FIGS. 11A-11B, 11C-11D, and 11E-11F, respectively) and fluorescence detection using the optical detection module (FIGS. 11G and 11H).

FIGS. 11A,11C, and 11E performed thermal lysis for 5 min at 95° C., and FIGS. 11B,11D, and 11F performed chemical lysis for 20 min at room temperature. In FIGS. 11A-11H, PC (positive control) represented 5 × 10⁴ copies/reaction of synthesized RNAs for each gene, NC (negative control) represented nuclease-free water, L represented 50-bp DNA ladder and lanes/columns 1 to 4 represented 5 × 10¹ to 5 × 10⁴ copies/reaction of extracted RNA from inactive viruses. In FIGS. 11G and 11H, error bars represented the standard error of the mean (n=3). ** was indicated as p<0.05 by two-tailed student t test analysis.

The photographs of gels in electrophoresis showed that using thermal lysis or chemical lysis arrived similar results that the LOD for the RdRp gene was about 5×10² copies/reaction and the LOD for the E gene and N gene were about 5 × 10³ copies/reaction. In addition, the fluorescence results (FIGS. 11G and 11H) corroborated this gel-based finding.

### Test Example 6: On-Chip Sensitivity Tests Using Clinical Samples

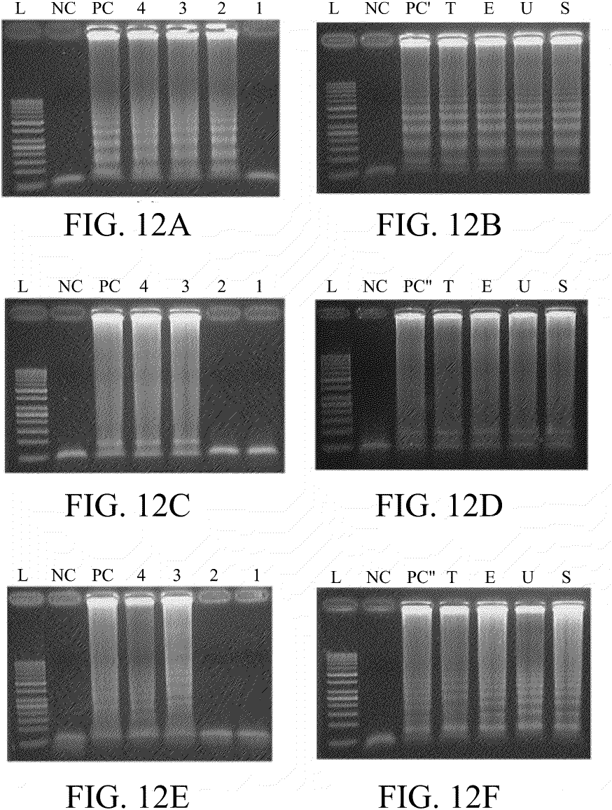

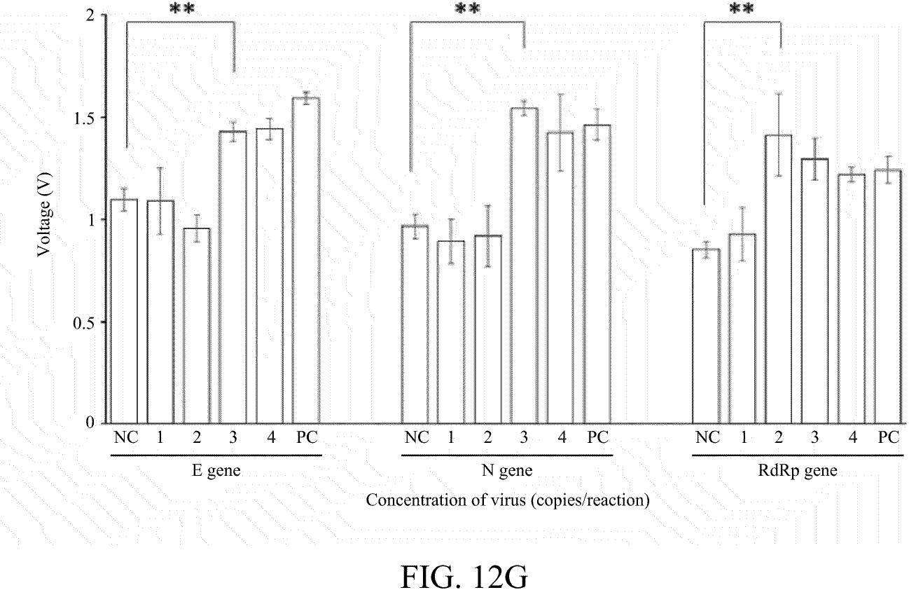

Test Example 6 was sensitivity tests of RT-LAMP using SARS-COV-2 RNA (initial concentration is 10⁸ copies/µL) provided by NCKU hospital, which is extracted from clinical COVID-19 patients infected in Taiwan, England, USA, and Spain. The results of sensitivity tests for the RdRp gene, E gene, and N gene were shown by gel electrophoresis (FIGS. 12A-12B, 12C-12D, and 12E-12F, respectively) and fluorescence detection using the optical detection module (FIG. 12G).

In FIGS. 12A-12G, NC (negative control) represented nuclease-free water and L represented 50-bp DNA ladder. In FIGS. 12A, 12C, 12E, and 12G, PC (positive control) represented 5 × 10⁴ copies/reaction of synthesized RNAs for each gene, and lanes/columns 1 to 4 represented 5 × 10¹ to 5 × 10⁴ copies/reaction of extracted RNA from the patient infected in Taiwan. In FIG. 12B, PC’ represented 5 × 10² copies/reaction of synthesized RNAs for the RdRp gene, and lanes T, E, U, and S represented 5 × 10² copies/reaction of extracted RNA from the patient infected in Taiwan, England, USA, and Spain, respectively. Similarly, in FIGS. 12D and 12F, PC’ represented 5 × 10³ copies/reaction of synthesized RNAs for the E gene and N gene, and lanes T, E, U, and S represented 5 × 10³ copies/reaction of extracted RNA from the patient infected in Taiwan, England, USA, and Spain, respectively. In FIG. 12G, error bars represented the standard error of the mean (n=3). ** was indicated as p<0.05 by two-tailed student t test analysis.

All results no matter in gel electrophoresis or fluorescence detection showed that LOD of the integrated microfluidic device of the present disclosure was excellent and achieved a very low detection concentration of 5 × 10² copies/reaction for the RdRp gene, and 5 × 10³ copies/reaction for the E gene and N gene. In addition, the integrated microfluidic device of the present disclosure can detect three genes simultaneously, thereby reducing false positive or negative. Moreover, the integrated microfluidic device can be used to test more samples at the same time through the design of flow channels and chambers, for example, the flow channels and chambers as shown in FIG. 6A can be designed into multiple sets and integrated into one chip.

### Example 2: An Integrated Microfluidic Device with a Pneumatic Combined Electromagnetic Control Module

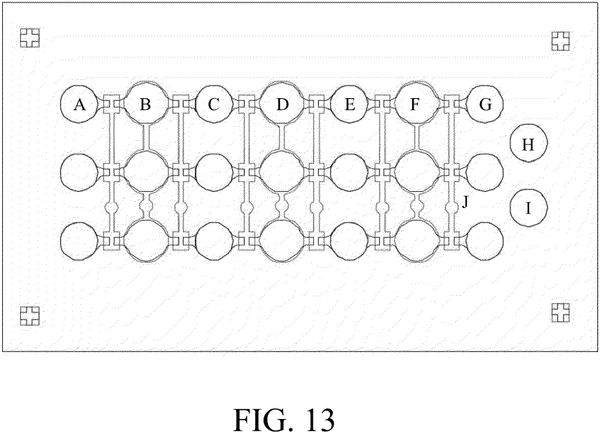

The other example of the microfluidic chip was shown in FIG. 13, which was composed of three layers: an air channel layer, a liquid control layer, and a substrate layer. The microfluidic chip was also made by pouring PDMS into PMMA molds, curing and bonding to glass substrate as Example 1, but the patterns, such as the configuration and numbers of channels, chambers, and holes, on the chip were different therefrom. In Example 2, the microfluidic chip comprises micropumps, microvalves, channels, chambers, and air injection holes. The microfluidic chip was a 70 × 42 × 10 mm chip having five parallel chambers for simultaneous detection of three genes, a positive control, and a negative control.

The microfluidic chip was placed on the platform comprising a flow control module, a temperature control module, and optionally further comprising optical detection module. The flow control module was a pneumatic combined electromagnetic control module (see FIG. 3B). the pneumatic control part comprised a compressor (TC-10, Centenary Material Co., Ltd., Taiwan), a vacuum pump (DC-18V-12, UNi-CROWN Co., Ltd., Taiwan), and 2 regulators (IR1000-01B-A for the compressor and IRV10-C06B for the vacuum pump, SMC Inc., Japan), and the electromagnetic control part comprised electromagnetic valves (EMVs, SMC S070B-SBG-05, Wei-Chia Eletro Material, Taiwan) controlled by Arduino microcontroller (Italy). The pneumatic control part provided a positive gauge pressure and a negative gauge pressure for actuating the micropumps and shutting normally-closed microvalves on chip. On the other hand, the air passage was controlled by switching on/off the electromagnetic valve (EMV) of the electromagnetic part.

Unlike the magnetic flow control module in Example 1, both samples and reagents here were transported by pneumatically driven membrane-type micropumps, which also functioned as micromixers. By providing the positive and negative gauge pressures to the air injection holes, the micropumps and microvalves could control the liquid mixing and transport. A positive gauge pressure was provided by closing the microvalves and activating the micropumps, and a negative gauge pressure was provided by opening the microvalves.

For liquid transport, the liquid was first stopped by a closed microvalve via a positive gauge pressure. The microvalve was opened via a negative gauge pressure to allow the liquid to pass, and at the same time, the liquid was drawn into a chamber having a micropump due to the suction produced by the micropump via a negative gauge pressure. Then, the microvalve was closed again via a positive gauge pressure, and a microvalve on the other side was opened via a negative gauge pressure, causing the liquid to leave the chamber and enter a channel or another chamber. Under this mechanism, liquid transport was achieved.

For mixing, after the liquid was drawn into a chamber, the chamber communicated with the other chamber by opening the microvalve between the two via a negative gauge pressure, and the micropumps set in the two drove the liquid to reciprocate between the two chambers by alternatively providing a positive gauge pressure and negative gauge pressure to the micropumps.

The temperature control module based on FIG. 4 comprises 2 TE coolers (TEC1.127.10, Tande, Taiwan) and 2 10-mm-thick copper plates (Scientific Instrument Center, NTHU, Taiwan) were placed on the top of TE coolers for thermal homogeneity. The temperature sensors (Max6675, Centenary Materials Co., Ltd., Taiwan) were inserted in the middle of the copper plates to monitor the temperature. The microcontroller is used as the analog to digital converter (Arduino Uno, Arduino, Italy) to control the TE coolers.

The integrated microfluidic device optionally further comprises the optical detection module (FIG. 5). The optical detection module comprised PMT, a light source (Diode-Pumped Solid-State laser, MBL-473, CNI, China), a set of filters and reflectors, an objective lens, and a microcontroller. The output wavelength of this laser was 473 ±1 nm and the emission wavelengths of the fluorescence in the present disclosure were 520 ±10 nm. The optical detection module further comprised a laser blocker for the control laser illumination and the implemented switch for the laser blocker was a stepper motor (MG995, TowerPro, Taiwan). The motor would drive the light blocker to control the incidence of the laser beam. The optical detection module was mounted with the bandpass filters (for excitation wavelength: 470±10nm; for emission wavelength: 500 to 550 nm, Mirle, Taiwan) for fluorescence detection. Then, the signals were detected by the PMT (C6271+R928, HAMAMATSU, Japan) along with detection time. Eventually, all the signals were collected by the microcontroller (Arduino Uno, Arduino, Italy).

Before detection, all materials were first prepared and loaded on the microfluidic chip. Table 6 below lists materials pre-loaded on the microfluidic chip when using virus samples.

TABLE 6

Materials loaded on the microfluidic chip

| | | |
|---|---|---|
| Chamber | Materials and volume | Source |
| A | 35 µL Lysis buffer | R145, ABP Biosciences, USA |
| | 35 µL Sample | |
| C | 5 µL Magnetic beads coated with RNA probes (coated probes SEQ ID NO. 13-15 in each row) | 10⁵ beads/µL, 1.05 µm, Invitrogen, USA |
| E | 30 µL Washing buffer | |
| G | 24µL RT-LAMP composition (for the RdRp gene, E gene, and N gene in each row) | |
| H | 25 µL Positive control | Antech Diagnostics, USA |
| I | 25 µL Negative control | |

As shown in Table 6, in at least one embodiment of the present disclosure, the process is as follows:
1. The Viral RNA lysis buffer (R145, ABP Biosciences, USA, 35 µL) was loaded into chambers A in each row;
2. MyOne™ carboxylic acid magnetic Dynabeads (1.05 µm, Invitrogen, USA) were coated with the RNA probes of SEQ ID NO. 13-15 (modified by amine group) respectively, wherein the coating was based on carboxylation with 1-ethyl-3-(3-dimethylaminopropyl) carbodiimide catalytic action, and these magnetic beads were then loaded into chambers C in each row (10⁶ beads/µL, 5 µL/chamber);
3. The washing buffer (nuclease-free water, 30 µL) was loaded into chambers E in each row;
4. The RT-LAMP composition (24 µL/chamber) as shown in Table 4 was loaded into chambers G in each row (for fluorescence detection, 1 µL of 0.5 mM calcein (C0875, Sigma-Aldrich) was added to the RT-LAMP composition and the volume of nuclease free water was adjusted to 9.2 µL);
5. Synthesized RNAs for RdRp gene, E gene, and N gene (Antech Diagnostics, USA, 10¹³ copies/µL, 1 µL) and the RT-LAMP composition (24 µL/chamber) were loaded into chamber H as positive control;
6. The nuclease free water (25µL) was loaded into chamber I as negative control; and
7. After the above loading, the sample (35 µL), selected from synthesized RNAs (for the RdRp gene, E gene and N gene, Antech Diagnostics, USA), synthesized cDNA, inactive viruses (Qnostics, UK) or RNAs extract from clinical samples (NCKUH, Taiwan) was loaded into chambers A in each row.

In Example 2, chambers B, D, and F in each row were chambers having micropump and were not preloaded materials. Holes J were the air injection holes.

During the detection, virus samples (e.g. inactive viruses, Qnostics, UK) were firstly lysed in chamber A for 20 minutes at room temperature or for 5 minutes at 95° C. through the temperature control module. The lysis product was transported to chamber C to mix with magnetic beads and then transported back to chamber A. The mixing was then performed by reciprocating between chamber A and chamber B. The mixture was transported to chamber C and RNA extraction was carried out via the magnetic beads for 10 minutes at 45° C. An external magnet was placed under the chambers to attract and collect the magnetic beads, and the liquid waste was discarded. The washing buffer was then added from chamber E to chamber C to wash the magnetic beads. The magnetic beads were attracted and collected by the external magnet followed by discarding the washing buffer. After that, RT-LAMP compositions were transported from chamber G to chamber C to mix with the magnetic beads. Finally, the mixture was transported back to chamber G to perform RT-LAMP for 60 minutes at 60° C. The RT-LAMP composition loaded in chambers G in each row respectively comprises a combination of the forward outer primer (F3), the backward outer primer (B3), the forward inner primer (FIP), and the backward inner primer (BIP) for the RdRp gene, E gene, and N gene. Dumbbell-shaped RT-LAMP products were produced.

The protocol of the detection for virus samples on the microfluidic chip were shown in Table 7 below.

TABLE 7

Protocol of the detection on Example 2

| Stages | Steps and duration | Reagents and volume | Position | Operating conditions | |
|---|---|---|---|---|---|
| | | | | Temperature | Gauge pressure (kPa) |
| Viral lysis | Thermal lysis for5 min or Chemical lysis for 20 min | 35 µL Sample + Lysis buffer | A | Thermal lysis at 95° C. Chemical lysis at room temperature | 10/-40 |
| RNA extraction | a. Addition of beads | Lysis product + 5 µL beads | A ➔ C ➔ A | | 10/-40 |
| | b. Mixing repeated multiple times) | | A ➔ B ➔ A | | 10/-15 |
| | c. Transport | | A ➔ C | | 10/-15 |
| | d. RNA extraction for 10 min | | C | 45° C. | 10/-40 |
| | e. Collection of beads and liquid waste removal | External magnet placed under the chambers | C ➔ A | | 10/-15 |
| Washing | a. Transport of washing buffer | 35 µL washing buffer | E ➔ C | | 10/-15 |
| | b. Liquid waste removal | | C ➔ A | | 10/-15 |
| RT-LAMP | a. Addition of RT-LAMP composition | 24 µL RT-LAMP composition + Magnetic beads | G ➔ C ➔ G | | 10/-40 |
| | b. RT-LAMP for 60 min | 24 µL RT-LAMP composition + Magnetic beads | G | 60° C. | 10/-40 |

On the other hand, for RNA samples (e.g. synthesized RNAs and RNAs extracted from clinical samples), the protocol was to directly perform RT-LAMP (and optionally RNA isolation). The following Test Example 7 to 11 were used Example 2.

### Test Example 7: On-Chip RT-LAMP Specificity Tests Using Synthesized RNAs

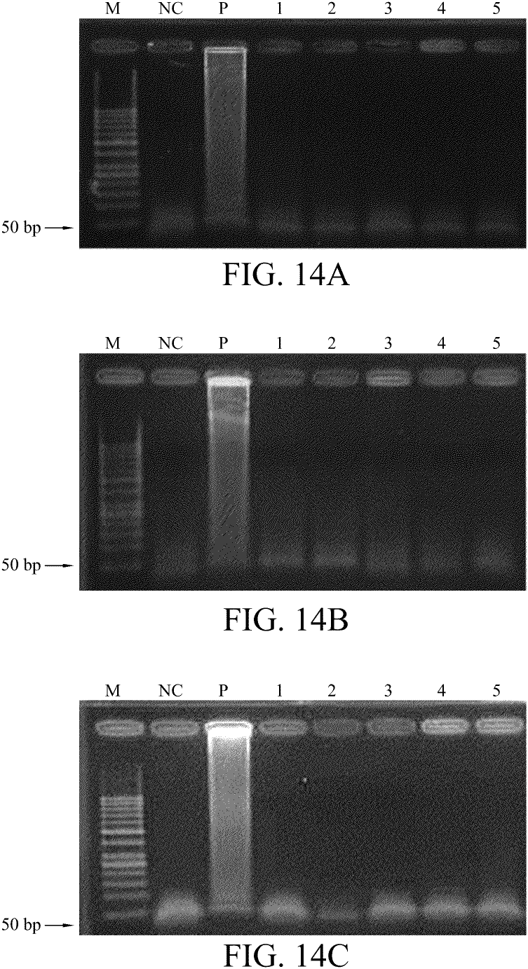

The specificity of the RT-LAMP assay was explored using synthesized RNA samples, as well as common acute upper respiratory viruses and bacteria. On-chip RT-LAMP specificity tests for the E gene, N gene, and RdRp gene were shown in FIGS. 14A-14C, respectively.

In FIGS. 14A-14C, lanes 1 to 5 of gel electrophoresis represented the RT-LAMP products (10¹¹ copies/reaction) from samples of Streptococcus pneumonia, Mycobacterium bovis, Influenza A virus, Influenza B virus, and Klebsiella pneumonia, respectively. P (positive control) represented the RT-LAMP products (10³ copies/reaction) from synthesized RNA of SARS-CoV-2. NC (negative control) represented the RT-LAMP products from nuclease free water and M represented 50-bp DNA ladder.

The results showed that the designed primers only reacted with the synthesized RNA of SARS-CoV-2, and the E gene, N gene, and RdRp gene of SARS-CoV-2 could be successfully amplified, indicating that the designed primers exhibited satisfactory specificity.

### Test Example 8: On-Chip Sensitivity Tests Using Synthesized RNAs

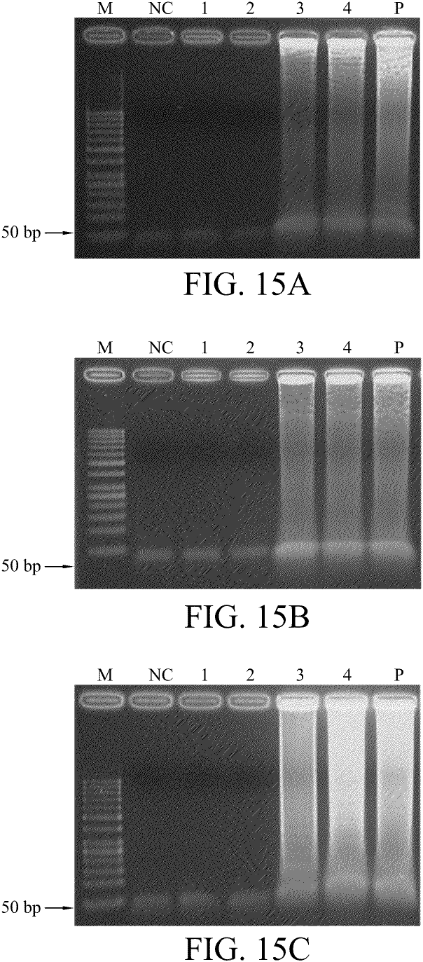

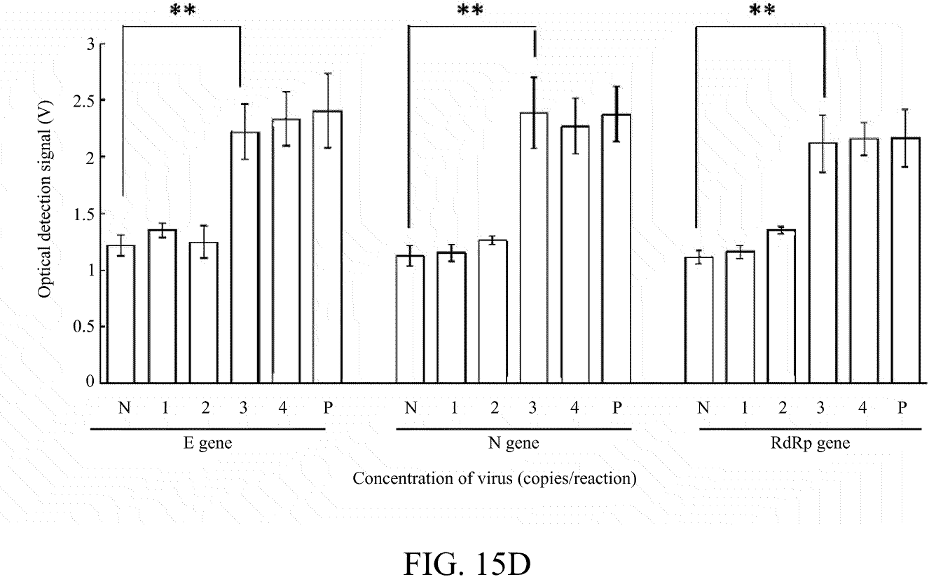

Next, E gene, N gene, and RdRp gene constructs from synthesized RNAs of SARS-CoV-2 serially diluted from 5 × 10⁴ to 5 × 10¹ copies/reaction were used to explore the sensitivity of RNA extraction + RT-LAMP on-chip (FIGS. 15A-15D).

FIGS. 15A-15C were the results shown by gel electrophoresis for E gene, N gene, and RdRp gene, respectively, and FIG. 15D was a plot of RT-LAMP optical detection signal (for fluorescence) versus starting concentrations. Lanes/columns 1 to 4 represented gene constructs from synthesized RNAs of SARS-CoV-2 at 5 × 10¹ to 5 × 10⁴ copies/reaction, respectively. P (positive control) represented gene constructs at 5 × 10⁵ copies/reaction. NC (negative control) represented the RT-LAMP products from nuclease free water and M represented 50-bp DNA ladder. Error bars represent the standard error of the mean (n=3). ** indicated as p<0.05 by the two-tailed student t test assay. The p values for the three genes were all calculated to be 0.0026.

In results of gel electrophoresis, each gene was successfully amplified after RNA extraction and RT-LAMP on chip. The LODs were found to be 5 × 10³ copies/reaction for the E gene, N gene, and RdRp gene when using synthesized RNA of SARS-CoV-2.

To evaluate the fluorescence intensities in real-time, the optical detection module was used to detect fluorescence every minute, and, after a 60-min reaction, fluorescence signals were evident and varied in intensity across differing starting concentrations. The two-tailed student t test showed that there was a significant difference (p<0.05) for LOD determination for 3 genes with a concentration higher than 5 × 10³ copies/reaction, which indicated that the optical detection was reliable.

### Test Example 9: On-Chip Sensitivity Tests Using Inactive Viruses

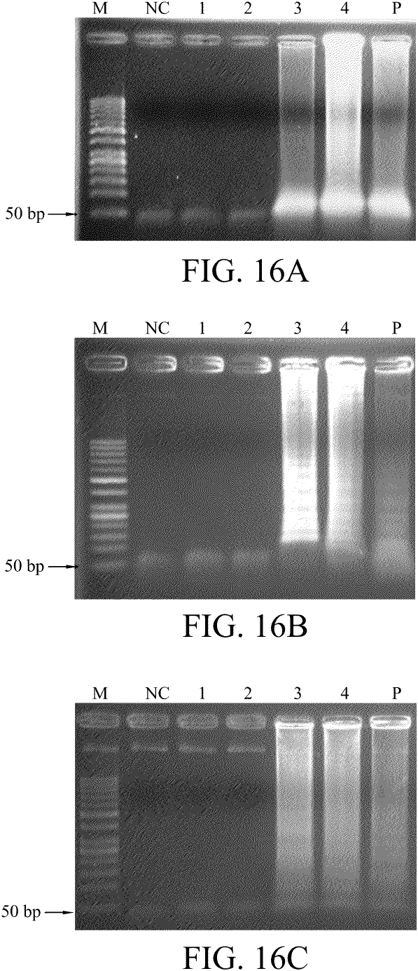

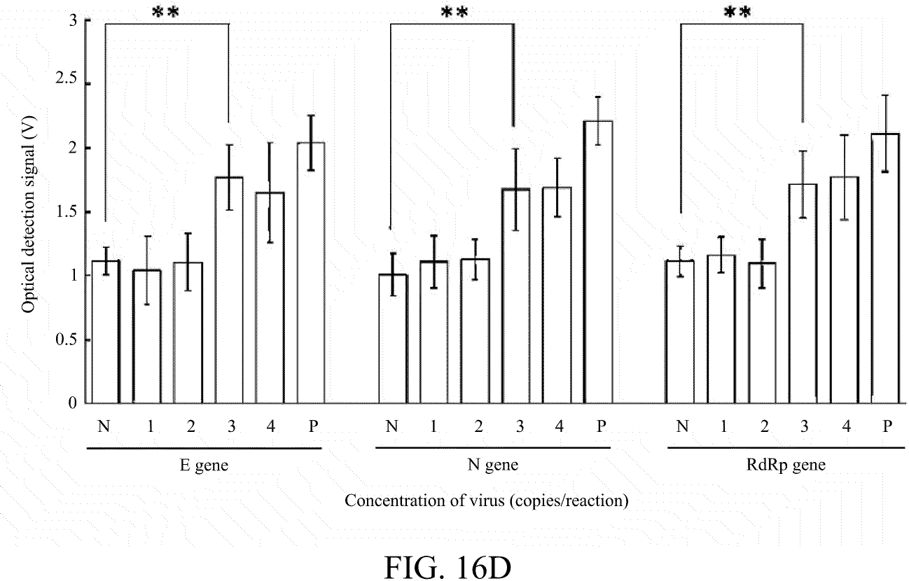

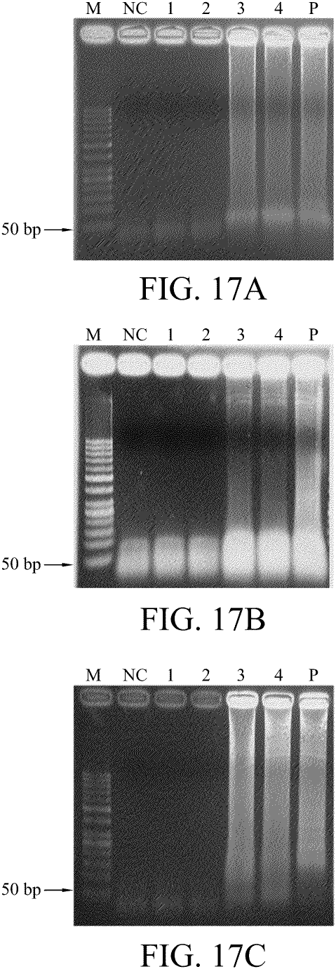

The integrated microfluidic device was then tested serially diluted inactive SARS-CoV-2 viruses (5 × 10⁴ to 5 × 10¹ copies/reaction), and each gene was successfully amplified after on-chip viral lysis + RNA extraction + RT-LAMP (FIGS. 16A-16D).

FIGS. 16A-16C were the gel electrophoresis results of viral lysis + RNA extraction + RT-LAMP on chip using inactive SARS-CoV-2 virus samples for the E gene, N gene, and RdRp gene, respectively. FIG. 16D showed a plot of RT-LAMP optical detection signal (for fluorescence) versus starting concentrations. Lanes/columns 1 to 4 represented gene constructs from extracted RNA of inactive SARS-CoV-2 viruses at 5 × 10¹ to 5 × 10⁴ copies/reaction, respectively. P (Positive control) represented gene constructs from inactive SARS-CoV-2 viruses at 5 × 10⁵ copies/reaction. NC (negative control) represented the RT-LAMP products from nuclease free water and M represented 50-bp ladder. Error bars represent the standard error of the mean (n=3). ** indicated as p<0.05 by the two-tailed student t test assay. The p values for the three genes were all calculated to be 0.0148, 0.033, and 0.0230, respectively.

Here, LODs were 5 × 10³ copies/reaction (i.e., 2 × 10² copies/µL) for all genes, which were significantly lower than the prior art. In addition, the integrated microfluidic device of the present disclosure can detect the E gene, N gene, and RdRp gene simultaneously. The corresponding fluorescence detection results also highlighted the high performance in sensitivity. The two-tailed student t test revealed a significant difference (p<0.05) for LOD determination for each gene with a concentration higher than 5×10³ copies/reaction. Hence, the optical detection could be conducted accurately.

### Test Example 10: On-Chip Sensitivity Tests Using Clinical Samples

Clinical samples of lineage B. 1.1.7. (i.e., the original COVID-19 virus) were used to test the sensitivity of the integrated microfluidic device of the present disclosure. The clinical samples were provided by NCKUH and loaded on the chip to carry out viral lysis, RNA extraction, and RT-LAMP.

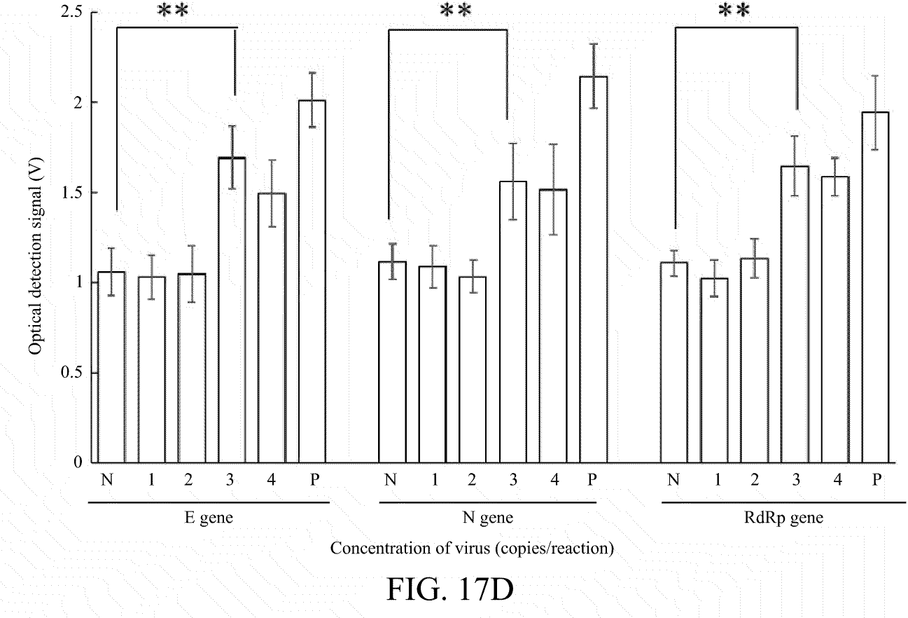

FIGS. 17A-17C were the gel electrophoresis results for the E gene, N gene, and RdRp gene, respectively. FIG. 17D showed a plot of RT-LAMP optical detection signal (for fluorescence) versus starting concentrations. Lanes/columns 1 to 4 represented gene constructs from extracted RNA of inactive SARS-CoV-2 viruses at 5 × 10¹ to 5 × 10⁴ copies/reaction, respectively. P (Positive control) represented gene constructs from extracted RNA of inactive SARS-CoV-2 viruses at 5 × 10⁵ copies/reaction. NC (negative control) represented the RT-LAMP products from nuclease free water and M represented 50-bp ladder. Error bars represent standard error of the mean (n=3). ** indicated p<0.05 by the two-tailed student t test assay. The p values for the E gene, N gene, and RdRp gene were determined to be 0.0293, 0.0063, and 0.0073, respectively.

All three genes were successfully amplified (FIGS. 17A-17D), and on-chip LOD of the integrated microfluidic device of the present disclosure was 5×10³ copies/reaction (i.e., 2 × 10⁵ copies/mL or Ct of 32.6) for each gene (the same as for synthesized RNA & inactive viruses). In the previous work, threshold (Ct) values >30 or copy numbers <10⁶ copies/mL were defined as “non-infectious.” By contrast, the integrated microfluidic device of the present disclosure has made great progress and can detect clinically meaningful levels of viruses. This is further evidenced in FIG. 17, in which fluorescence differences can be observed across dilutions. Moreover, as for each gene, there was a significant difference (p<0.05) for LOD determination in the two-tailed student t test. Therefore, the device could detect each gene.

### Test Example 11: Real-Time Quantification

During the 60-min RT-LAMP, signals were acquired every minute for real-time detection, and a threshold time could be defined as the time required for the fluorescence signal to cross over a threshold. Since calcein was used, the threshold time was inversely proportional to the amount of initial target in the sample. With this approach, real-time fluorescence signals could be used to quantify the initial concentration of viruses. The data were fitted to a sigmoidal curve, and the threshold time was calculated as the sum of the mean fluorescence signal during the initial 5 minutes and 5 times of the standard deviation values during the same time.

$$\begin{matrix}\text{Threshold} \text{value} = \text{mean} \text{of} \text{fluorescence} \text{signal} + 5 \times \\ \text{standard} \text{deviation} \text{of fluorescence} \text{signal}\end{matrix}$$

Therefore, the threshold time, which was inversely proportional to the initial amount of the target, could be determined by the point of intersection of the threshold value and the fitted curve.

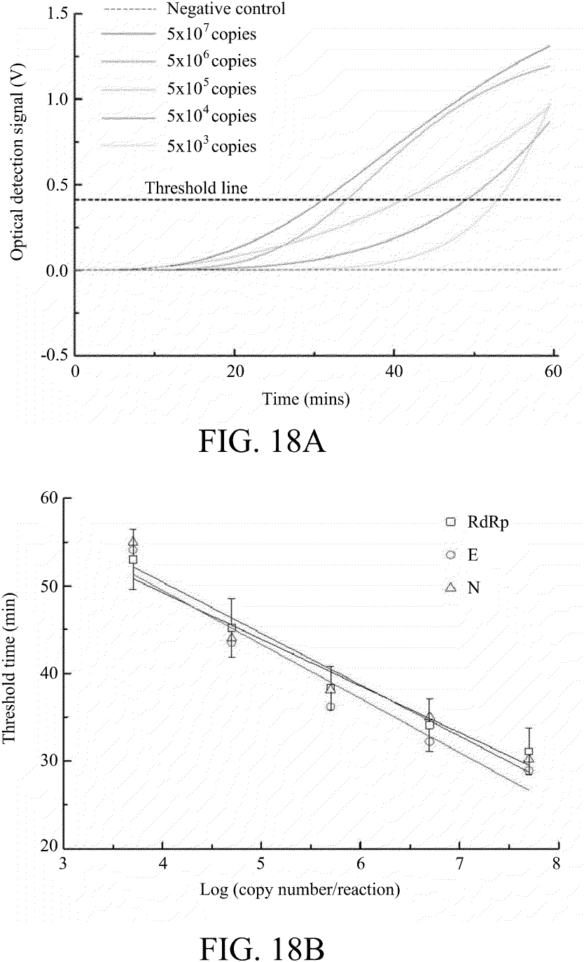

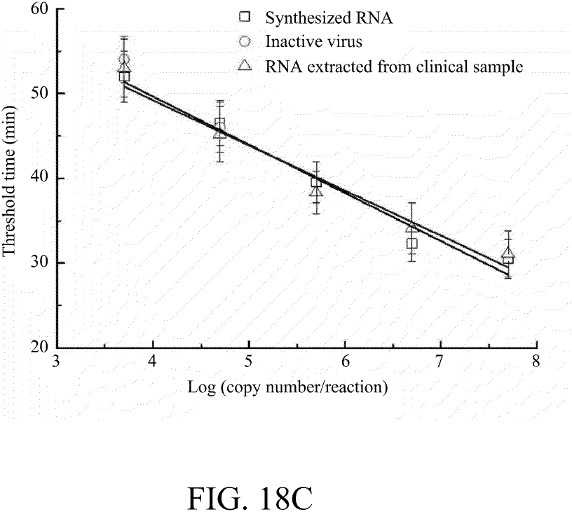

In Test Example 11, synthesized RNAs were prepared in concentrations ranging from 5 × 10⁷ to 5 × 10³ copies/reaction, with optical detection module monitored over time (FIG. 18A). FIG. 18B showed standard curves of the threshold time versus Log (copy number/reaction) for three genes using clinical samples. FIG. 18C showed standard curves of the threshold time versus Log (copy number/reaction) for the RdRp genes using synthesized RNA, inactive virus, and clinical samples. In these figures, error bars represented standard error of the mean (n=3).

Based on these results, all trends were relatively linear with similar slopes. It showed that the optical detection module was capable of real-time and automatic detection of virus molecules. Besides, the laser blocker in the optical detection module provided a shorter response time (2 sec) and could provide more reliable optical detection. The automatic integrated microfluidic device of the present disclosure achieved to detect the E gene, N gene, and RdRp gene, simultaneously, precisely, and accurately. The LOD is found to be 5×10³ copies/reaction (i.e. 2 × 10⁵ copies/mL) for all three genes. Moreover, these genes could also be detected in real-time within only 90 minutes. The integrated microfluidic device of the present disclosure could consequently revolutionize COVID-19 diagnostics.

All references cited and discussed in this specification are incorporated herein by reference in their entireties and to the same extent as if each reference was individually incorporated by reference.

### Gel Electrophoresis

20 µL of LAMP products were mixed with 5 µL of 6X DNA loading dye (Promega, USA, containing 0.4% orange G, 0.03% bromophenol blue, 0.03% xylene cyanol FF, 15% Ficoll® 400, 10 mM Tris-HCl [pH 7.5], and 50 mM EDTA [pH 8.0]) and then were loaded into 2% agarose (#0710-500G, VWR Life Science AMRESCO, USA) gels in 0.5x Tris-borate-EDTA (V4251, Promega) buffer- along with the 8.5 µL of 50 base-pair (bp) DNA ladders (AccuBand™ 50 bp DNA Ladder II, 55.5 µg/ 500 µl, (not consistency) DM1200; SMOBio, Hsinchu city, Taiwan). Samples were electrophoresed at 100 V for 35 min. After that, the gels were incubated in the ethidium bromide (0.5 µg/mL; 200 mL, Sigma, USA) solution for 10 min. The bands were visualized under ultraviolet (UV) transilluminator (BioDoc-ItTM Imaging System, UVP, Canada) with an exposure time of 0.5 to 1.2 sec.

What is claimed is:

1. An integrated microfluidic device for SARS-CoV-2 detection, comprising:
a microfluidic chip having a plurality of chambers for loading a sample, a reagent, a buffer, or a mixture thereof, wherein the chambers comprise:
a plurality of first functional chambers containing a loop-mediated isothermal amplification (LAMP) composition, wherein the LAMP composition in each of the first functional chambers comprises primers of SEQ ID NOs. 1 to 4, primers of SEQ ID NOs. 5 to 8, or primers of SEQ ID NOs. 9 to 12;
a flow control module for transporting the sample, the reagent, the buffer, or the mixture thereof between the chambers; and
a temperature control module for controlling and/or keeping a temperature during a reaction.

2. The integrated microfluidic device of claim 1, wherein the first functional chambers contain primers of SEQ ID NOs. 1 to 4, primers of SEQ ID NOs. 5 to 8, and primers of SEQ ID NOs. 9 to 12, and wherein the temperature during the reaction of LAMP is in a range of from 60° C. to 65° C.

3. The integrated microfluidic device of claim 1, wherein the chambers further comprise at least one second functional chamber for loading the sample and/or conducting viral lysis.

4. The integrated microfluidic device of claim 1, wherein the microfluidic chip further comprises at least one third functional chamber for RNA extraction, the third function chamber contains an RNA capture reagent coated with an RNA probe selected from the group consisting of SEQ ID NO. 13, SEQ ID NO. 14, and SEQ ID NO. 15, and the RNA capture reagent is a magnetic bead.

5. The integrated microfluidic device of claim 1, wherein the temperature control module comprises:
a thermoelectric cooler;
a relay configured to turn on the thermoelectric cooler for heating or to turn off the thermoelectric cooler for cooling; and
a thermocouple.

6. The integrated microfluidic device of claim 1, wherein the microfluidic chip further comprises:
a fourth functional chamber having a micropump for mixing; and
a microvalve arranged between any two adjacent ones of the chambers.

7. The integrated microfluidic device of claim 6, wherein the flow control module is a magnetic control module comprising a permanent magnet and an electromagnet respectively set on both sides of the micropump and the microvalve.

8. The integrated microfluidic device of claim 6, wherein the flow control module is a pneumatic combined electromagnetic control module comprising a vacuum pump, a compressor, and an electromagnetic valve, and wherein the microfluidic chip further comprises an air hole for air flow controlled by the pneumatic combined electromagnetic control module.

9. The integrated microfluidic device of claim 1, wherein the LAMP composition further comprises a fluorescent dye, and the integrated microfluidic device further comprises an optical detection module for exciting the fluorescent dye to generate a fluorescence signal and detecting the fluorescence signal.

10. The device of claim 9, wherein the optical detection module comprises a light source, an objective lens, and a photomultiplier tube.

11. A method for detecting SARS-CoV-2, comprising:
providing the integrated microfluidic device of claim 1;
loading a sample into the chambers; and
conducting the LAMP at a temperature of from 60° C. to 65° C.,
wherein the steps after loading the sample into the chambers are automatically operated by the flow control module and/or the temperature control module.

12. The method of claim 11, wherein the chambers further comprise at least one second functional chamber, and the sample is loaded into the second functional chamber, and wherein the method further comprises:
conducting viral lysis at room temperature or a temperature of from 90° C. to 100° C. in the second functional chamber to obtain a lysis product containing RNA;
dividing the lysis product into multiple parts;
mixing each of the multiple parts of the lysis product with the LAMP composition.

13. The method of claim 12, wherein the microfluidic chip further comprises at least one third functional chamber containing an RNA capture reagent, and wherein before mixing the lysis product with the LAMP composition, the method further comprises:
transporting the lysis product to the third functional chamber;
mixing the lysis product with the RNA capture reagent; and
conducting RNA extraction in the third chamber at a temperature of from 40° C. to 50° C.

14. The method of claim 13, the RNA capture reagent is a magnetic bead coated with an RNA probe selected from the group consisting of SEQ ID NO. 13, SEQ ID NO. 14, and SEQ ID NO. 15.

15. The method of claim 13, wherein the LAMP is conducted in the first functional chamber or the third functional chamber.

16. The method of claim 13, further comprising washing the RNA capture reagent after the RNA extraction.

17. The method of claim 13, wherein the flow control module is a magnetic control module comprising a permanent magnet and an electromagnet, and wherein the steps of transporting and mixing are controlled by turning on the electromagnet to create a magnetic attraction to the permanent magnet and/or turning off the electromagnet to cancel the magnetic attraction to the permanent magnet.

18. The method of claim 13, wherein the flow control module is a pneumatic combined electromagnetic control module comprising a vacuum pump, a compressor, and an electromagnetic valve, and wherein the step of transporting is controlled by producing a positive pressure and a negative pressure by the compressor, the vacuum pump, and the electromagnetic valve.

19. The method of claim 11, wherein the LAMP composition further comprises a fluorescent dye and the integrated microfluidic device further comprises an optical detection module, and wherein during or after the step of conducting the LAMP, the method further comprises:
exciting the fluorescent dye to generate a fluorescence signal; and
detecting the fluorescence signal by the optical detection module.

20. The method of claim 19, further comprising quantifying a concentration of the SARS-CoV-2 according to an accumulative curve of the fluorescence signal.
